(19) United States
(12) Patent Application Publication
(10) Pub. No.: US 2020/0407801 A1
(43) Pub. Date: Dec. 31, 2020

(54) Method for Generating a Profile of the DNA Repair Capabilities of Tumour Cells and the Uses Thereof

(71) Applicant: LXREPAIR, Grenoble (FR)

(72) Inventor: Sylvie Sauvaigo, Grenoble (FR)

(73) Assignee: LXREPAIR, Grenoble (FR)

(21) Appl. No.: 16/970,071

(22) PCT Filed: Feb. 26, 2019
(86) PCT No.: PCT/FR2019/050434

§ 371 Date: Sep. 2, 2020

(30) Foreign Application Priority Data
Feb. 27, 2018 (FR) 18 51724

(51) Int. Cl. C12Q 1/6886

(52) U.S. Cl. CPC C12Q 1/6886; C12Q 2600/112; C12Q 2600/106; C12Q 2600/118

(57) ABSTRACT

The invention relates to a method for generating a profile of DNA repair capacities of tumor cells and uses thereof for cancer prognosis, choice, monitoring and/or the prediction of the therapeutic efficacy of a cancer treatment in a patient, and also for screening anticancer drugs. The invention also relates to a reference library comprising profiles of DNA repair capacities for various subtypes of a cancer, obtained by the method of the invention, and to uses thereof for the classification of cancers.

The present invention relates to a method for generating a profile of DNA repair capacities of tumor cells and uses thereof, in particular for cancer prognosis, choice, monitoring and/or the prediction of the therapeutic efficacy of a cancer treatment in a patient, and also for screening anticancer drugs. The present invention also relates to a reference library comprising profiles of DNA repair capacities for various subtypes of a cancer, obtained by the method of the invention, and to the uses thereof for the classification of cancers.

Conventional cancer treatments (e.g. surgery, radiation therapy, chemotherapy) rest on protocols suited to each cancer depending on the stage of progression and the cancer type, determined according to standard classification criteria such as the TNM classification (Tumor, Node, Metastasis). Immunotherapy for cancers, which revolutionized the care for patients in therapeutic failure, comprises the use of monoclonal antibodies, in particular inhibitors of immune checkpoints such as anti-CTLLA-4, anti-PD-1 and anti-PD-L1, and other bispecific antibodies which bind cancer cells and immune cells.

In order to optimize therapeutic care for patients, multiple biomarkers are developed for stratification (or classification) of patients at different stages of their care, in particular for diagnosis, prognosis, choice, monitoring and prediction of the therapeutic efficacy of a treatment.

Although it is indispensable, stratification of cancer patients is very complicated and involves the use of numerous biomarkers. This is because of the heterogeneity of each of the cancer types and subtypes and the complexities of the mechanisms of appearance of cancers and treatment resistance. This complexity and its difficulties are shown by some examples.

Cancers are in particular classified into various subtypes according to the expression of markers by tumor cells. For example, breast cancers are classified in subtypes according to the expression of estrogen receptors (ER) and progesterone receptors (PR) and HER2/ERB2/neu. Generally, the ER+ and PR+ cancers are treated with tamoxifen. However, not all ER+ breast cancers respond to treatment and other markers are sought for identifying nonresponders. Triple-negative breast cancers (ER−, PR−, ERB2−) are treated with chemotherapy but failures and relapses are frequent. Many biomarkers are being explored for characterizing these subtypes, but for now none has shown clinical usefulness.

Omics techniques have been used for characterizing breast cancers at the DNA, RNA, protein and carbohydrate level (Judes et al., Cancer Lett., 2016, 382, 77-85; WO 2001/075160). These markers are not sufficiently robust and precise to be clinically useful.

Cancers are also classified in various subtypes by the presence of genomic alterations in the tumor DNA such as mutations in certain key genes, chromosomal rearrangements or gene amplifications, at the origin of activation of proliferation pathways and cellular survival. For example, melanomas are classified in subtypes according to mutations of various key genes (BRAF, NRAS), responsible for constitutive activation of the MAP kinase pathways. However, the use of specific inhibitors directed against mutated BRAF proteins, for example, is not always associated as expected with the inactivation of the signaling pathways (Manzano et al., Ann. Transl. Med., 2016; 4, 237). The resulting classification is therefore not sufficient for identifying responders and does not allow administering good treatment to patients.

Cancers are also classified according to genome alterations in the tumor DNA (e.g. mutations, mutation rates, microsatellite instability (MSI), gene amplification) supporting the appearance of neoantigens on the tumors, which could predict the response to immunotherapies. Just the same, DNA amplification technologies used for identifying mutations at the origin of neoantigens introduce biases and errors and are not reliable for this application (“The problem with neoantigen prediction,” Nat. Biotech., 2017, 35, 97). In general, methods relying on the detection of genomic changes in tumor DNA do only a very limited analysis of the defects which could occur and especially do not study either the functional causes or consequences.

In addition to the preceding markers, defects in the DNA repair system are also used as biomarkers indicative of the development, progression and treatment response of cancers.

Six repair systems have been identified in humans, including two with the function of eliminating modified DNA bases. The Base Excision Repair (BER) system is more specifically dedicated to the repair of small lesions of DNA bases (e.g. oxidative damage, abasic sites, fragmentation, methylations and Etheno-bases). The Nucleotide Excision Repair (NER) system handles voluminous lesions inducing a distortion of the DNA double helix (e.g. acetylamino-fluorine, cisplatin and psoralen adducts; dimers induced by UV-B and UV-C; covalent lesions formed between one DNA base and another molecule). These two repair systems have shared characteristics and in all cases comprise: i) an excision step during which the modified nucleotide is eliminated; and ii) a resynthesis step which uses polymerases present in the environment during which at least one nucleoside triphosphate (nucleotide) present in the environment is incorporated as a replacement in the DNA strand.

Application US 2017/198,360 describes a molecular test with which to distinguish cancer subtypes according to the genetic defects in DNA repair leading to the over or under expression of transcription products related to the repair of the DNA. This stratification is used for selecting the most appropriate chemotherapies among drugs targeting DNA or DNA repair activities. However, it is well known that tumor resistance or sensitivity to treatments directly or indirectly affecting the DNA or the repair of the DNA arises from deregulation of DNA repair, but also from deregulation of the DNA damage response (DDR) signaling and from several molecular sensors and effectors regulating in their turn the DDR and belonging in particular to the cellular signaling/proliferation pathways. These complex networks bring redundant enzymatic networks into action, in particular regulated at the post-translational level or by protein-protein interactions. Thus, the assay of gene transcription products does not provide effective information on the repair activities actually functional or actually inactive. Further, some defects in the activities can be compensated by the action of various proteins/enzymes. These compensatory mechanisms cannot be detected genetically either. Thus, the proposed molecular test cannot determine precisely or with pertinence the functional DNA repair capacities in the tumors.

Consequently, the patient stratification methods proposed today are not sufficient and are very limited relative to the cancer subtype that they characterize. They do not allow selecting the most appropriate therapy from the choice of protocols proposed today, according to the therapeutic class of agents used or combinations of therapeutic agents from different classes. They do not allow identifying patients who are going to respond to various therapies.

Consequently, there is a need to have methods available for stratification of patients which are more effective.

DNA defects accumulate when the DNA repair systems are functionally deficient. These function deficiencies can be caused by dysfunctions at various levels of the molecular regulations governing DNA repair: epigenetic, DNA, RNA, proteins, post-translational modifications. It is therefore much more pertinent to look for how to characterize directly the functionality of the repair systems than the mutational status of some genes or zones of the genome chosen a priori.

Some studies have shown that there is an association between cancer types or subtypes and the repair capacity of the nuclear proteins from a tumor/a cancer (Forestier et al., PLoS One., 2012, 7, e51754). In particular, DNA repair activities are closely associated with cellular signaling/proliferation pathways and the functionality thereof. Mutations in the cellular signaling/proliferation pathways impact DNA repair capacities (Velic D et al, Biomolecules, 2015, 5, 3204-3259). DNA repair activities are closely associated with mutations, translocations, amplifications and instabilities of microsatellites. Repair capacities reveal repair defects which have consequences at the functional level.

Metastatic melanoma has long been the subject of a poor prognosis with chemotherapy and radiation therapy response rates of 15 to 20% (Quirt et al., Oncologist, 2007, 12, 1114-1123). DNA repair mechanisms could be responsible for this high failure rate (Sarasin A. and Dessen P., Curr. Mol. Med., 2010, 10, 413-418; Tentori et al., Curr. Med. Chem., 2009, 16, 245-257).

More generally, the efficacy of nucleotide excision repair modulates the response of patients to cisplatin (Gavande et al., Pharmacol. Ther., 2016, 160, 65-83) and the efficacy of radiation therapy depends on the capacities for double strand break repair (Biau et al, Neoplasia, 2014, 16, 835-844).

The international application WO 2004/059,004 describes a method for quantitative evaluation of overall and specific DNA repair capacities of at least one biological medium wherein DNA lesion repair by repair enzymes present in the biological medium to be tested is done in the presence of a single labeled nucleotide.

However, it is not possible to be in optimal conditions for studying the repair of each of the lesions and determining what specific pathway repaired it by using a single labeled nucleotide.

In fact, the various lesions are formed by chemical or physical reactions starting with very specific bases or nucleosides (C, G, T or A) and are repaired by pathways not leading to the simple replacement of the lesion but to the possible further replacement of nucleotides surrounding the lesion depending on the nature of the pathway taken. The BER can act by a “short patch” pathway (replacing 1 to 3 nucleotides) or a “long patch” pathway (replacing some 10 nucleotides); NER can also be involved and lead to the replacement of 25 to 30 nucleotides. Sometimes the NER mechanism can substitute for the BER mechanize.

If the repair of an L1 lesion takes a BER short patch pathway, it is mostly the nucleotide at the origin of the L1 formation that will be incorporated. Hence, if the repair reaction were to take place with a different labeled nucleotide, the repair would be poorly evaluated because it would be considered as ineffective even though it hadn't been quantified under the appropriate conditions.

If the repair reaction of the L1 lesion were to take place with the corresponding labeled nucleotide but not with other nucleotides, it wouldn't be possible to compare the efficacy of incorporation of the various nucleotides and it wouldn't be possible to say whether the repair was defective or not.

If the repair of an L1 lesion were to take the BER long patch pathway or the NER pathway, there would be statistically identical incorporation of all the nucleotides. Performing the reaction with at least two different nucleosides is indispensable for verifying it.

The use of a single nucleotide can lead to false interpretations, not give information on the fidelity of the repair and not allow conclusions as to the patch excision repair mechanism elicited.

There is no method in the prior art with which to establish a precise profile of the DNA repair capacities of a tumor.

The Applicant has developed a method for generating a profile of DNA repair capacities of tumor cells with which to remedy these disadvantages.

Thus, the present invention relates to an in vitro method for generating a profile of DNA repair capacities of tumor cells, comprising at least the following steps:

a) incubating a tumor cell extract comprising a DNA repair activity with at least two damaged DNA molecules comprising distinct DNA lesions and at least two different labeled nucleotides;

b) measuring the quantity of each labeled nucleotide incorporated in each damaged DNA molecule resulting from the activity of the repair enzymes present in said cellular extract in step a);

c) determining, from the values measured in step b), at least one of the following parameters:
c1) the enzymatic signature of the DNA repair;
c2) the contribution of the repair of each DNA lesion independently for each labeled nucleotide compared to the total repair; and
c3) the relative rate of incorporation of each labeled nucleotide for at least two DNA lesions and independently for each DNA lesion; and

d) establishing the profile of DNA repair capacities of said tumor cells based on the parameters determined in step c).

In accordance with the present invention, “tumor cells” is understood to mean tumor cells isolated from a biological sample collected from a patient and the tumor cell lines. Tumor cells are in particular isolated from a tumor sample or a blood sample containing circulating tumor cells, collected from the patient. The tumor sample is for example obtained by biopsy or surgical resection of the tumor. The tumor cells are generally isolated by dissociation of the tumor tissue, in particular by enzymatic digestion, for example with collagenase. This preparatory step is not necessary when the biological sample contains circulating tumor cells.

In accordance with the present invention, “tumor” is understood to mean a malignant tumor, meaning a tumor associated with a cancer. A tumor includes a primary tumor and a metastasis. The metastasis can be located in a lymph node draining the tumor (nodular metastasis) and/or in a different tissue or organ from the primary tumor.

The cancers that can be classified by the method ofthe invention in particular include cancers related to exposure to genotoxic agents like melanoma, in particular metastatic melanoma (related to ultraviolet radiation exposure), lung cancer (related to tobacco exposure) and cancer of the head and neck (related to tobacco and alcohol exposure); other cancers such as breast cancer, ovarian cancer, endometrial cancer, sarcomas; or any other solid cancer for which a biopsy or invaded node is available.

In accordance with the present invention, “previously characterized tumor” means a tumor characterized by conventional methods for classification of tumors and possibly new molecular biology methods or any other method with which to establish tumor categories.

Conventional tumor classification methods in particular include: anatomical-pathological and histological classification which considers the organ or tissue of origin and the histological type of the tumor and serves to determine the type of cancer; the classification in grades which serves to evaluate the degree of differentiation of the tumor and its progression (slow and local or rapid); the TMN (Tumor, Node, Metastasis) which serves to determine the stage of progression of the cancer.

New tumor classification methods are based on a division of tumors in subtypes, depending on indicative biomarkers, such as genetic, epigenetic, proteomic, and glycomic markers, imaging or other biomarkers. Biomarkers indicative of the presence of a cancer, in particular markers specific to certain tumor types, are diagnostically useful. Biomarkers indicative of the future development of a cancer (progression, prediction and monitoring of treatment response), are useful for the prognosis, treatment selection, monitoring and prediction of the therapeutic efficacy of a treatment. The following can be given as examples of biomarkers: genes, RNA, proteins, metabolites or biological processes.

Cancer types can in particular be characterized or classified in various subtypes based on markers expressed by tumor cells. For example, breast cancer is divided into subtypes according to the expression of estrogen receptors (ER) and progesterone receptors (PR) and HER2 by the tumor cells.

Cancer types can also be characterized or classified in various subtypes by the presence of genomic alterations in the tumor DNA such as mutations in certain key genes, chromosomal remnants or gene amplifications, at the origin of activation of proliferation pathways and cellular survival. These genomic alterations are in particular merged genes, deletions, mutation hotspots, gene variants, high mutation levels, genes with multiple copies (gene amplification), microsatellite instability (MSI) markers, mutations in key genes known for being responsible for activation of proliferation signaling pathways and oncogenesis, or anti-oncogenes, and combinations of these genomic alterations. The following can be given as examples of the preceding mutations: mutations in DNA repair genes, DNA damage response, tyrosine kinase activity receptors, kinases, in particular EGFR, BRAF, PIK3CA, AKT1, ERBB2, PTEN, MEK, MAP2K1, KIT, CDKN2A, MYC, or anti-oncogenes like TP53. For example, in the context of metastatic melanoma classification, the cancer subtypes are mutated BRAF, mutated NRAS, and unmutated BRAF and NRAS.

Cancers are also classified according to genome alterations in the tumor DNA (e.g. mutations, mutation rates, microsatellite instability, gene amplification) that favor the appearance of neoantigens on the tumors and could predict the response to immunotherapies.

Genomic alterations are disclosed by “omic” methods (e.g. genomic, proteomic, transcriptomic, metabolomic) or any other method with which to directly or indirectly characterize the genomic alterations. The various cancer subtypes do not react the same way to recommended therapeutic treatments.

In accordance with the present invention, “patient” is understood to mean an individual, human or animal, preferably human, with cancer.

“Treatment” is understood to mean an antitumor or anticancer therapeutic treatment. In particular it is a matter of chemotherapy, radiation therapy, targeted therapy and immunotherapy.

In accordance with the present invention, “plasmid” is a supercoiled plasmid, meaning without single-stranded or double-stranded break of the DNA.

“Repair” is understood to mean the repair of the DNA; “repair signature,” the signature of DNA repair capacities; “repair profile,” the profile of DNA repair capacities.

“Damaged DNA,” “damaged DNA molecule,” “lesioned DNA” or “lesioned DNA molecule” are understood to mean a DNA molecule comprising DNA lesions, preferably a single type of DNA lesions or several lesions created by a single genotoxic or chemical agent.

In an embodiment of the invention, said tumor cells are isolated from a tumor sample or from blood from the patient.

According to an advantageous disposition of this embodiment, said tumor cells are isolated from at least one previously characterized tumor.

In particular, the tumor is defined by conventional tumor classification criteria such as defined above. Further, the tumor is defined by one marker of interest for the diagnosis and/or treatment of this type of cancer, in particular a biomarker or combination of specific biomarkers of the subtype of this cancer, which could be associated with one or more biomarkers of the progression, prediction and/or monitoring of the response to a treatment of this cancer type or subtype.

The characterized tumor corresponds in particular to the subtype of the cancer of the patient to be tested or for which new antitumor agents or a more effective treatment are sought.

The implementation of the method of the invention on tumor cells isolated from characterized tumors such as described above serves to obtain a repair profile of tumor cells which is specific to a specific cancer subtype. Such a repair profile which is associated with a specific cancer subtype is named reference repair profile or reference profile.

The method of the invention is advantageously implemented with a set of different tumors from a single type of previously characterized cancer (tumor library or bank), so as to get a set of reference profiles called reference library or bank. Preferably, the method of the invention is implemented with tumors corresponding to various subtypes of one type of cancer including at least the cancer subtypes of patients to be tested, in particular the cancer subtypes which orient or condition the choice of treatment in patients, so as to get specific reference profile banks for various subtypes of various types of cancer in particular cancer subtypes which condition the choice of the treatment in patients. For each of these particular cancer subtypes, the reference bank preferably includes the reference profile of patients responding and/or not responding to the recommended therapeutic treatment, in particular a targeted therapy, in particular therapy guided by mutations in key genes, like for example the mutations in BRAF and NRAS genes from melanoma.

In an embodiment of the invention, the patient has a cancer linked to exposure to genotoxic agents, for example a melanoma, in particular metastatic melanoma of determined subtype, specifically, mutated BRAF, mutated NRAS or unmutated for BRAF and NRAS.

For implementation of the method of the invention, isolated tumor cells are pretreated so as to extract nuclear proteins which contain the DNA repair enzymes. The extraction of nuclear proteins from isolated tumor cells is done according to standard techniques well known to the person skilled in the art. The isolated tumor cells are generally lysed for isolating the nuclei and then the nuclei are in turn lysed for extracting nuclear proteins. In order to prepare the extracted nuclear proteins, the protocols described for in vitro transcription tests or in vitro DNA repair tests can be used, such as for example those described by Dignam JD et al. (Nucleic Acids Research, 1983, 11;1475-1489); Kaw L. et al. (Gene Analysis Techniques, 1988, 5, 22-31); Iliakis G. et al. (Methods Mol. Biol., 2006, 314, 123-31); Luo Y et al. (BMC Immunol., 2014, 15, 586-). The nuclear extracts can further be fractionated or dialyzed according to conventional methods. All the steps for preparation of the nuclear protein extract are done under conditions that do not denature the enzymatic activities and the proteins contained in the tumor cells, such that the DNA repair activity initially present in the sample of tumor cells is preserved in the cellular extract which is analyzed in the method of the invention.

In an embodiment of step a) from the method of the invention, the tumor cell extract comprising DNA repair activity is a cellular lysate, preferably a nuclear lysate, and in a preferred way an extract of nuclear proteins from said tumor cells.

According to the method of the invention, the damaged DNA which serves as repair matrix for the DNA repair enzymes present in the tumor cell extract is a single- or double-stranded DNA whether linear or circular, short (under 100 bases) or long (over 100 bases). In particular it involves short or long DNA fragments such as those described in the application WO 01/090408 or a supercoiled circular double-stranded DNA, in particular a plasmid such as described in the Application WO 2004/059004, Millau et al., Lab. Chip., 2008, 8, 1713-1722; Prunier et al., Mutation Research, 2012, 736, 48-55. Preferably, the damaged DNA is a damaged plasmid.

According to the method of the invention, the damaged DNA comprises lesions induced by a known genotoxic physical or chemical agent, in particular by treatment of an isolated plasmid or of cells comprising said plasmid with a genotoxic agent. Among the lesions present in the damaged or lesioned DNA, the following can be particularly mentioned: lesions of purine and pyrimidine bases, sugars, double helix structure, and single-stranded and double-stranded breaks.

The lesions of purine and pyrimidine bases include:
abasic sites (AbaS), in particular depurination which can be generated by acid treatment of DNA at a high temperature as described in Millau et al. and Prunier et al., op. cit.;
thymine and/or cytosine glycols (Glycol), which can be generated by treatment of the DNA with potassium permanganate as described in Millau et al. and Prunier et al., 2008, op. cit.;
the alkylation, in particular the formation of etheno-bases (Etheno), such as etheno-guanine and/or etheno-adenosine, in particular etheno-guanine, induced in particular by trans,trans-2,4-decadienal (DDE), as described in the application WO 2004/059004, Millau et al. and Prunier et al., op. cit.; or the etheno-guanine and etheno-adenosine mixture created by chloroacetaldehyde (B. Tudek, et al. IARC Sci Publ (1999) 279-93).
oxidative lesions, in particular the formation of 8-oxo-guanine (8-oxo-G) or 8-oxo-2′-deoxyguanosine (8-oxo-dG); 8-oxo-G is in particular induced by photosensitization in the presence of riboflavin, as described in Millau et al. and Prunier et al.; the formation of 8-oxo-dG is in particular induced by treatment with an endoperoxide such as DHPNO2 as described in the Application WO 2004/059004;
photoproducts, in particular cyclobutane type pyrimidine dimers (CPD) and pyrimidines-pyrimidiones (6-4) (6-4 photoproducts or 6-4PP), alone or in mixture, induced by UV-B or C as described in the Application WO 2004/059004; Millau et al. and Prunier et al., op. cit.; the pyrimidine dimers formed by photosensitization to UV-A in presence of norfloxacin, as described in Sauvaigo, S., et al, Photochem. Photobiol. 2001; 73, 230-237;
chemical adducts, induced in particular by polycyclic aromatic hydrocarbons (PAH) such as benzopyrene; alkylating agents such as cisplatin, mitomycin and chlorambucil; aromatic amines; and mycotoxins;
deamination;
methylation; and
demethylation.

The lesions of the double helix structure include the formation of intra-strand and inter-strand bridges, generally caused by ultraviolet radiation and bifunctional antitumor agents, such as UV-B, cisplatin, psoralen in presence of UV-A, mitomycin and chlorambucil; the inserting agents form stable covalent bonds between opposite bases of the DNA strands. The lesions from cisplatin, mostly G-G and G-A intra-strand bridges, and G-G inter-strand bridges, are in particular generated as described in Millau et al. and Prunier et al., op. cit. The lesions from psoralen, in particular T-T intra-strand and inter-strand bridges, are generated by photosensitization to UV-A, as described in Millau et al.

The lesions from sugars (deoxyriboses) lead to a breakage of the phosphodiester bonds near the lesioned sites, followed by a breakage of the DNA strand.

Single-strand and double-strand breakages are produced by agents such as ionizing radiation and by the action of free radicals.

Preferably, damaged DNA comprises lesions of the purine and/or pyrimidine bases or lesions of the double helix structure such as defined above, meaning lesions which do not result in single-strand or double-strand breakage of the DNA.

In accordance with the method of the invention, each damaged DNA, preferably a damaged plasmid, carries predefined lesions, distinct from the lesions of another damaged DNA. Consequently, the lesions of each damaged DNA are characterized prior to implementation of step a), meaning the nature or the type of lesions and the number of lesions present on each DNA are determined as described in the Application WO 2004/059004; Millau et al. and Prunier et al., op cit.

Further, the supercoiled fraction of each plasmid is selected after treatment of the DNA by the genotoxic agent or the combination of genotoxic agents which induce lesions of the purine and/or pyrimidine bases and/or of the structure of the double helix such as defined above, so as to exclude strand breakages (relaxed structure) and avoid the action of nucleases.

In an advantageous embodiment of the method of the invention, the step a) is performed with at least two damaged DNAs comprising distinct lesions, preferably selected from the group consisting of:

1) 8-oxo-G;

2) Abasic sites (AbaS);

3) Etheno-bases (Etheno), in particular Etheno-guanines (Etheno-G) and/or Etheno-adenines (Etheno-A), in particular Etheno-G;

4) Thymine and/or cytosine glycols (Glycols); and

5) Photoproducts, in particular cyclobutane type pyrimidine dimers (CPD) and pyrimidines-pyrimidiones (6-4) (6-4 photoproducts or 6-4PP), alone or in mixture.

The damaged DNAs are advantageously damaged plasmids.

Preferably, each damaged DNA carries a single type of lesion such as listed above, specifically: 8-oxo-G, AbaS, Etheno, Glycols or Photoproducts.

In an advantageous embodiment of the invention, the step a) is performed with at least one first DNA, preferably a plasmid, comprising a single type of lesion repaired by the base excision repair (BER) system and at least one second DNA, preferably a plasmid, comprising a single type of lesion repaired by the nucleotide excision repair (NER) system.

Preferably the lesions repaired by the BER are chosen from the group consisting of of: 1) 8-oxo-G; 2) Abasic sites (AbaS); 3) Etheno-bases (Etheno), in particular Etheno-guanines (Etheno-G) and/or Etheno-adenines (Etheno-A), in particular Etheno-G; 4) Thymine and/or cytosine glycols, and the lesions repaired by NER are photoproducts, in particular cyclobutane type pyrimidine dimers (CPD) and pyrimidines-pyrimidiones (6-4) (6-4 photoproducts or 6-4PP), alone or in mixture, preferably in mixture.

According to an advantageous disposition of the preceding embodiment, the step h) is implemented with at least one first DNA, preferably a plasmid, comprising 8-oxo-G lesions and at least one second DNA, preferably a plasmid, comprising cyclobutane type pyrimidine dimers (CPD) and cyclobutane type pyrimidine dimers (CPD) and pyrimidines-pyrimidiones (6-4) (6-4 photoproducts or 6-4PP), alone or in mixture, preferably in mixture.

Preferably, step a) is performed with:
a first DNA, preferably a plasmid, comprising 8-oxo-G lesions;
a second DNA, preferably a plasmid, comprising abasic sites (AbaS);
a third DNA, preferably a plasmid, comprising Etheno-bases (Etheno), in particular Etheno-guanines (Etheno-G) and/or Etheno-adenines (Etheno-A), in particular Etheno-G;
a fourth DNA, preferably a plasmid, comprising thymine and/or cytosine glycols;
a fifth DNA, preferably a plasmid, comprising cyclobutane type pyrimidine dimers (CPD); and
a sixth DNA, preferably a plasmid, comprising a mixture of cyclobutane type pyrimidine dimers (CPD) and pyrimidines-pyrimidiones (6-4) (6-4 photoproducts or 6-4PP).

Preferably, the damaged or lesioned DNA (step a) is immobilized on a solid support, with which to easily eliminate the labeled nucleotides not incorporated in step a), by a washing step, before step b) of measuring incorporation of the nucleotide in the damage DNA. The solid support is an appropriate support for the immobilization of nucleic acids and in particular DNA, such as for example a support of glass, polypropylene, polystyrene, silicone, metal, nitrocellulose or nylon optionally modified by a porous film, in particular a nylon or hydrogel film, for example a polyacrylamide hydrogel. In particular it involves a glass or silicone slide covered with hydrogel. The support is advantageously a microchip type miniaturized support. Such supports modified by a porous film are in particular described in the Application WO 2006/136686; Millau et al. and Prunier et al., op. cit. To immobilize the DNA on the support, the DNA is deposited on the support, for example by automation with a robot such as a piezoelectric robot.

The method of the invention can be implemented in parallel on several tumor cell extracts, with one support on which different lesioned DNA series are immobilized. In particular it involves a partially or wholly automated, high rate method.

In accordance with the method of the invention, the nucleosides used as labels are conventional nucleosides: adenosine, cytidine, guanosine, thymidine or uracil, in triphosphate form, meaning nucleotides. The method operates with at least two triphosphate nucleosides (nucleotides) from dCTP, dGTP, dATP and dUTP.

The use of dGTP serves to produce more intense signals than with the other dNTP for the repair of lesions repaired by BER and formed from the guanine base, like for example 8-oxo-guanine. The use of another dNTP serves as a point of comparison and makes it possible to confirm that mostly dGTP is incorporated compared to other dNTPs.

The use of dATP serves to get more intense signals for the repair of lesions repaired with BER and created from the adenine base as for example etheno-adenine and the abasic sites. The use of another dNTP serves as a point of comparison and makes it possible to confirm that mostly dATP is incorporated in a compared to other dNTPs.

The abasic sites are also formed from depurination of the guanosine; it is thus interesting to compare the incorporation of dATP, dGTP and another dNTP for the repair of this lesion.

The etheno-base lesions are generally made up of a mixture of Etheno-A and Etheno-G, the use of a nucleotide other than dATP and dGTP serves as a point of comparison and makes it possible to confirm that the dATP and dGTP are incorporated in a larger quantity than the other dNTP.

The use of dCTP serves to get more intense signals for the repair of lesions repaired with BER and created from the cytosine base like for example cytosine glycols.

The use of dUTP serves to get more intense signals for the repair of lesions repaired with BER and created from the thymine base as for example thymine glycols.

The use of a dNTP other than dCTP or dUTP serves as a reference.

The use of at least two different dNTPs serves to assure that the repairs of photoproducts in fact operates by the NER pathway. Although the formation of photoproducts occurs especially near T and C, their repair, which passes through the elimination and replacement of some 30 bases, is going to lead to statistically equivalent incorporation of two nucleotides.

The repair aberrations measured by comparison of the signatures obtained in the presence of different nucleotides serves to identify the samples at the dysfunctional repair pathways.

Preferably, the step b) of the method of the invention is implemented with at least the labeled dGTP and dCTP nucleotides, preferably the labeled dCTP, dGTP, dATP and dUTP nucleotides. dCTP is preferred for the choice, monitoring, prediction of therapeutic efficacy of a targeted therapy, guided by the presence of mutations in key genes like the mutations in the BRAF and NRAS genes for melanoma.

The nucleotides are labeled, which makes it possible to detect and quantify them when they are incorporated in the damaged DNA following the DNA repair reaction. The tracer or label which is used for labeling nucleotides is detected by measurement of the signal which is proportional to the quantity of nucleotide incorporated in the damaged DNA, in particular the damaged plasmids.

The means and techniques for labeling nucleotides are well known to the person skilled in the art and include radioactive, magnetic, fluorescent, colorimetric or other labeling, which can be done directly or indirectly. The tracers or reagent for direct labeling are in particular radioactive isotopes or luminescent compounds (e.g. radioluminescent, chemiluminescent, bioluminescent, florescent or phosphorescent), such as the fluorophores, like, without limitation, Cyanine 3 and Cyanine 5. Indirect labeling agents include in particular affinity molecules such as biotin (streptavidin/biotin system), digoxigenin or any molecule which can be revealed by a ligand-receptor interaction, in particular with the help of an antibody or by a chemical treatment.

Labeling of dNTP, in particular fluorescent, radioactive, magnetic or colorimetric labeling is detectable by any technique known to the person skilled in the art such as, without limitation, fluorescence microscopy, flow cytometry, scintigraphy, magnetic resonance imaging and mass spectrometry.

The use of different labels makes it possible to do a single repair reaction per damaged DNA (step a) but requires a specific detector for each label (step b). The use of a single label for the various nucleotides requires doing the repair (step a) in separate reactions for each nucleotide, with each damaged DNA, but makes it possible to use a single detector for measuring the repair signal (step b).

Preferably, the label is a direct or indirect fluorescent label. Preferably, the nucleotides are labeled with a fluorophore. Even more preferably, all the nucleotides are labeled by the same fluorophore.

The repair reaction (step a) is implemented under conditions allowing the repair of DNA lesions by repair enzymes present in said cellular extract and the incorporation of each labeled nucleotide in each damaged DNA. These conditions, which are well known to the person skilled in the art, are described in the Application WO 2004/059004; Millau et al. and Prunier et al., 2008, op. cit. The repair reaction is done in the presence of ATP, an ATP regeneration system and any other agent necessary for the activity of DNA repair enzymes present in the tumor cell extract. For example, the repair buffer contains 200 mM Hepes/KOH, 7.8 pH; 35 mM MgCl₂; 2.5 mM DTT; 1.25 μM of each of the four dNTP; 1 mM ATP, 17% glycerol; 50 mM phosphocreatine; 10 mM EDTA; 250 μg/mL creatine phosphokinase and 0.5 mg/L BSA. The repair reaction is generally done with an extract of nuclear proteins containing 0.2 to 2 mg of proteins per mL. The reaction is done at a temperature favoring the repair reaction, preferably 30° C., for sufficient time, generally included between one and five hours. The reaction is advantageously done in the microwells of a microchip type miniaturized support such as defined above.

According to the invention, the repair reactions (step a) are done in parallel with a control DNA, not lesioned, which serves to measure the background noise of the repair reaction (step b). Consequently, the step a) further comprises control reactions for each labeled nucleotide, in which the cellular extract is incubated with a control DNA, not damaged and, separately, with each labeled nucleotide.

In advance of the step b) of measurement of the signal from incorporation of the label in the lesioned DNA (repair signal), the support on which the DNA is fixed is generally washed at least once using a saline solution containing a non-ionic surfactant, in particular a 10 mM phosphate buffer, containing 0.05% of Tween 20, and then is next rinsed with water at least once.

In accordance with the invention, the step b) comprises measurement of the incorporation signal of the label in the lesioned DNA, preferably the lesioned plasmid, for various repair reactions from the step a). Each reaction from step a) corresponds to the repair of DNA comprising a single lesion type such as defined above with a single labeled dNTP such as defined above.

Further, step b) also comprises the measurement of the incorporation signal of the label in the unlesioned control DNA, for each of the nucleotides labeled in step a), so as to measure the background noise of the repair reaction for each of the labeled nucleotides.

Measurement of the signal is done by a method suited to the label, using an instrument suited to the support and the label used. For example, if the label is a fluorophore, fluorescent signals emitted by the various deposits (or spots) of the support are measured directly. A scanner could be used for analysis of the image and fluorescence. Preferably an apparatus capable of exciting the fluorophore and measuring the signal emitted by the excitation is used.

The enzymatic signature of the repair of DNA from tumor cells (step c1)), coming in particular from a tumor from a patient, corresponds to the set of signals measured in step b) for the repair of all lesions present (n total lesions) by the extract of said tumor cells, with each labeled nucleotide (at least two nucleotides X and Y, and possibly a third nucleotide Z and a fourth nucleotide W).

The repair signal obtained with the nucleotides X and Y is the set:

[(I_{X1}-I_{C/X}); (I_{X2}-I_{C/X}); . . . ; (I_{Xn}-I_{C/X}); [(I_{Y1}-I_{C/Y}); (I_{Y2}-I_{C/Y}); . . . ; (I_{Yn}-I_{C/Y})].

where:

X is the first nucleotide for which the repair signal of all the lesions is being determined;
I_{X1} is the measured signal value for the nucleotide X against a first lesion;
I_{X2} is the measured signal value for the nucleotide X against a second lesion;
I_{Xn} is the measured signal value for the nucleotide X against an n^{th} lesion;
I_{C/X} is the measured signal value for the nucleotide X with the control plasmid;

Y is the second nucleotide for which the repair signal of all the lesions is being determined;
I_{Y1} is the measured signal value for the nucleotide Y against a first lesion;
I_{Y2} is the measured signal value for the nucleotide Y against a second lesion;
I_{Yn} is the measured signal value for the nucleotide Y against an n^{th} lesion;
I_{C/Y} is the measured signal value for the nucleotide Y with the control plasmid.

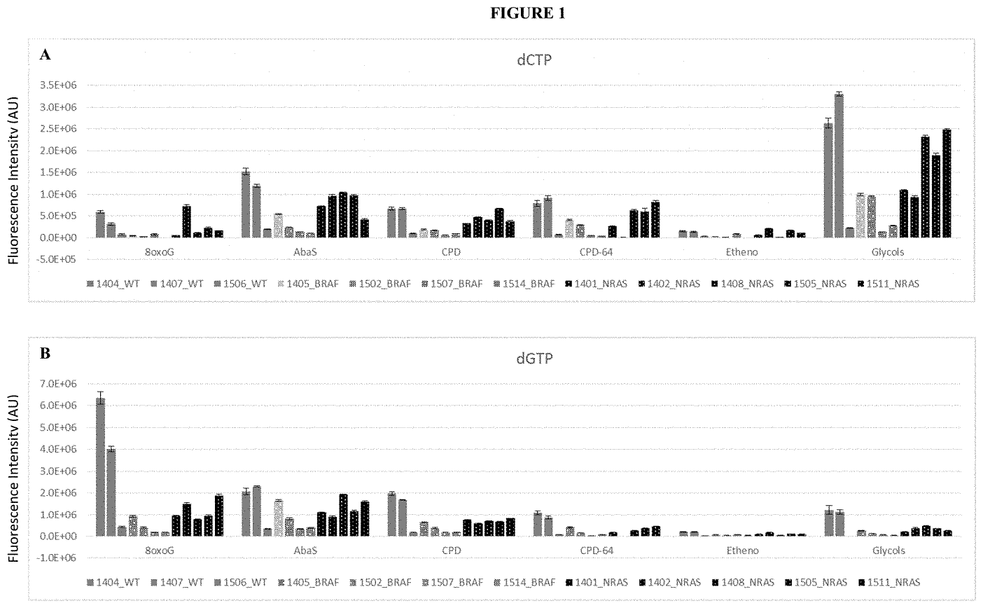

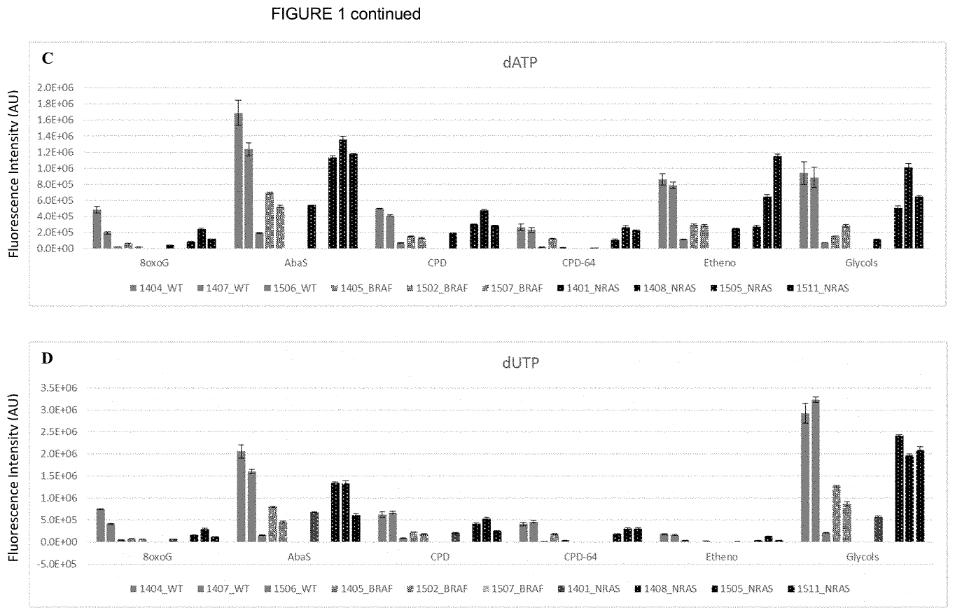

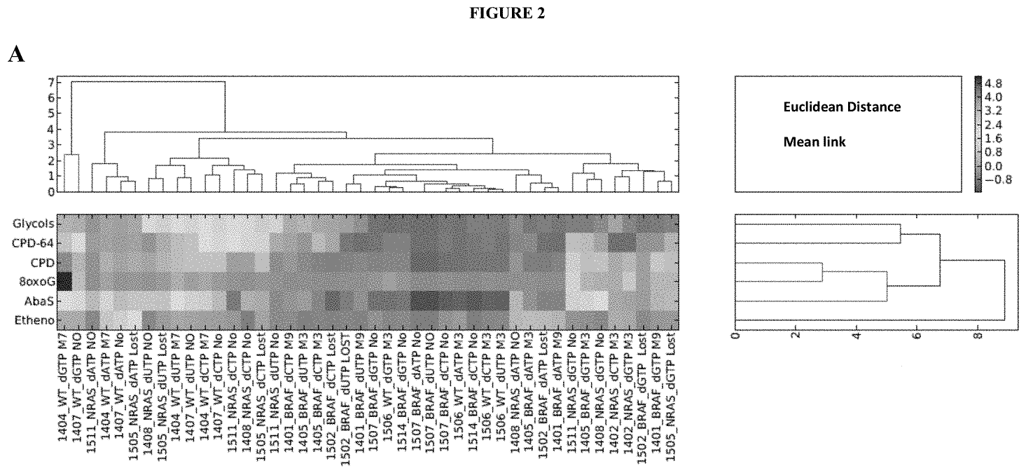

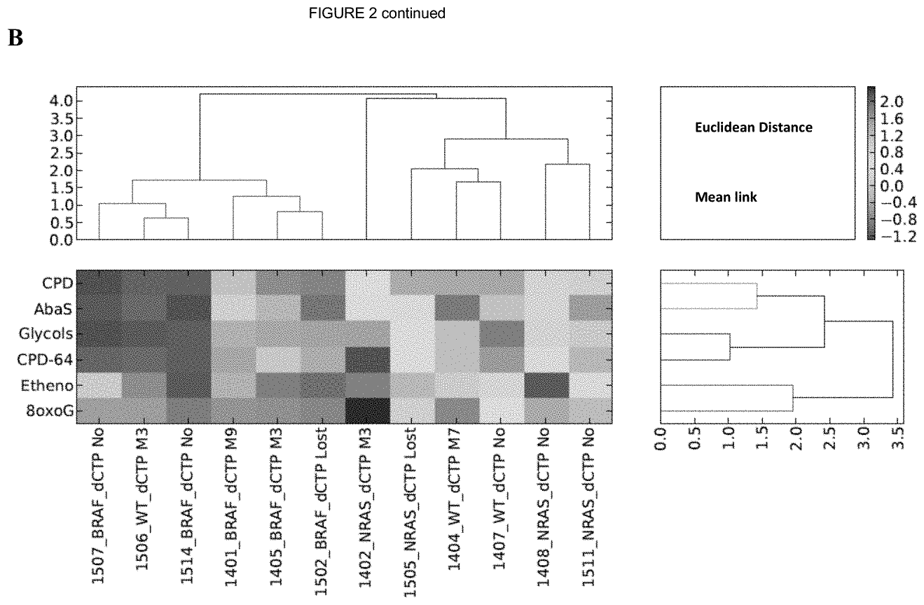

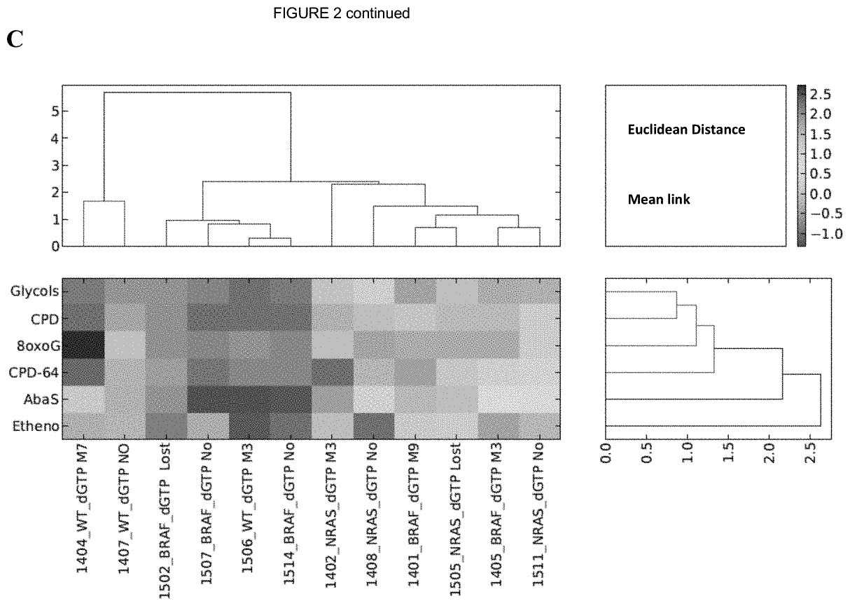

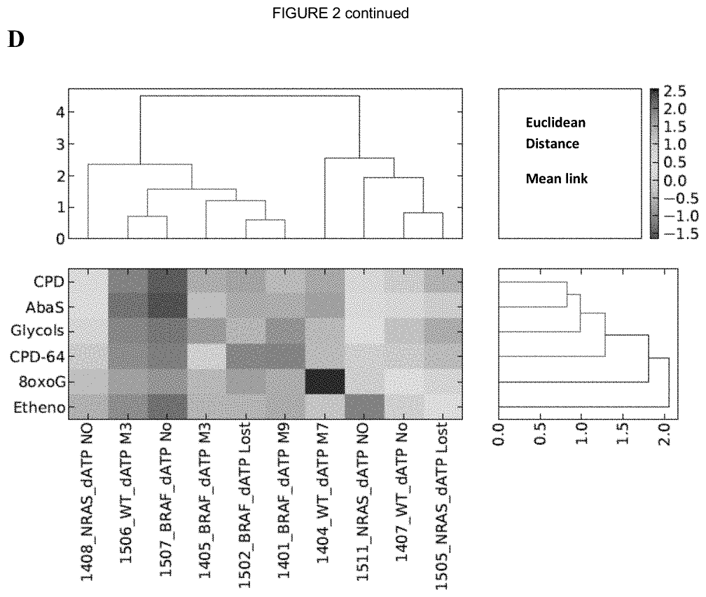

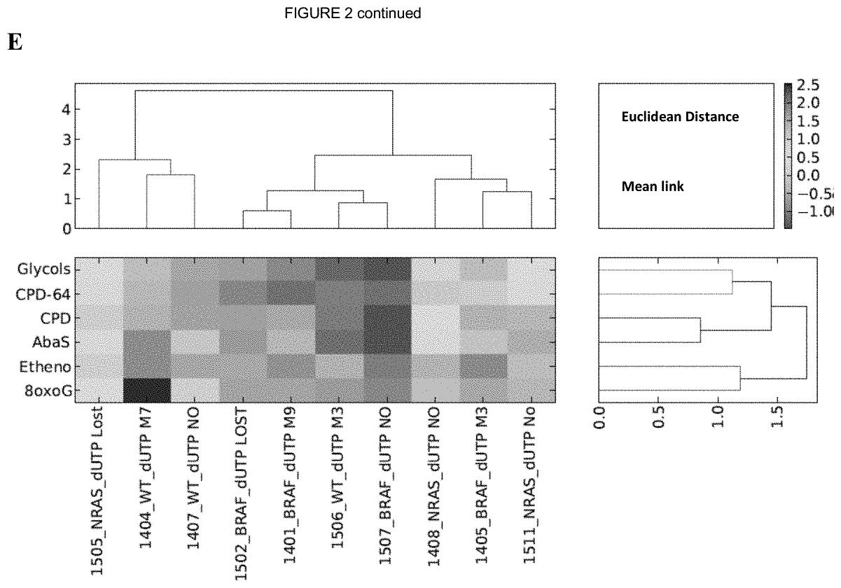

For each tumor cell sample, in particular isolated from a tumor sample from a patient, the resulting repair signal is different and depends on the labeled nucleotide used (FIG. 1A to 1D). It is analyzed by conventional classification methods (“clustering”) which take into account all of the signals obtained for a given sample for comparison of the samples with each other and against a reference library (FIG. 2A to 2E). The repair signature serves to characterize the repair capacities of the sample globally and essentially compares the intensity values obtained. By using at least two different nucleotides, the sample can be compared to the reference library obtained under each of the conditions and the consistency of the classification can be confirmed or disagreements uncovered. For example, a sample may be in a certain class defined by the repair signature from the library with the nucleotide X (corresponding to a subtype) and in another class defined by the repair signature with the nucleotide Y, which discloses an inconsistency in the classification. This inconsistency indicates that the sample is different from other samples from the class and therefore might not respond to the therapy prescribed for this class.

This first analysis is particularly suited for identifying responders to targeted therapies prescribed on the basis of known mutations. In fact, signatures of repair activities are closely associated with cellular signaling/proliferation pathways and functionality thereof. By comparison with reference profiles from reference libraries, it will be determined whether the profile from the tumor bearing one or more mutations guiding the prescription of targeted therapies corresponds to the profile bearing the mutation which responds to the treatment. If discrepancies appear, this indicates that the targeted therapy will not be effective.

With the repair signature obtained, samples can also be identified in which the repair activities for a given lesion are absent; this absence can be partial if the signal is measured with at least one nucleotide, or total if it is not measured with any nucleotide. The absence of some activities revealed by the method is going to characterize tumors presenting major genomic mutations or defects which could respond more favorably to immunotherapy.

The repair signatures are closely associated with defects of DNA repair mechanisms. They themselves are responsible for the sensitivity and resistance to chemotherapy and radiation therapy. The repair signatures can serve to determine the best therapeutic strategy to follow among treatments by DNA repair inhibitors, chemotherapy and radiation therapy, or combinations.

The different values obtained in step b) undergo different classifications or calculations, combined or not: repair signature, contribution, relative incorporation rate of each nucleotide, in order to get a repair profile giving the most information possible on the repair capabilities of the repair proteins from the tumor.

The repair profile distinguishes the different functional repair mechanisms while also quantifying them. More specifically, with this profile it can be distinguished whether:

tumors of a single type or subtype of cancer, determined on the basis of a key gene mutation, or on the basis of other markers determining the choice of a treatment, are ranked in a single group (are similar) or in a different group (no similarity);

at least one repair activity treating a given lesion is actually functional;

a given lesion is repaired by several repair activities;

a given lesion is repaired by base excision repair (BER) or nucleotide excision repair (NER);

when a lesion is repaired by BER, it is the short patch repair or the long patch repair which operates and therefore which polymerase is involved (beta or delta/epsilon);

the repair is faithful;

based on the repair signature obtained in step c₁), the contribution of the repair of each of the lesions compared to the total repair can be determined independently for each of the labeled nucleotides step c₂).

For example, the contribution (percentage) of the repair of the first lesion relative to the total repair (n lesions), for the nucleotides X and Y is determined, as a percentage, by following the formula:

$$Contribution for X = \frac{{I}_{X 1} - {I}_{C / X}}{Σ ( ( {I}_{X 1} - {I}_{C / X} ) + ( {I}_{X 2} - {I}_{C / X} ) + ( {I}_{Xn} - {I}_{C / X} ) )} \times 100$$

$$Contribution for Y = \frac{{I}_{Y 1} - {I}_{C / Y}}{Σ ( ( {I}_{Y 1} - {I}_{C / Y} ) + ( {I}_{Y 2} - {I}_{C / Y} ) + ( {I}_{Yn} - {I}_{C / Y} ) )} \times 100$$

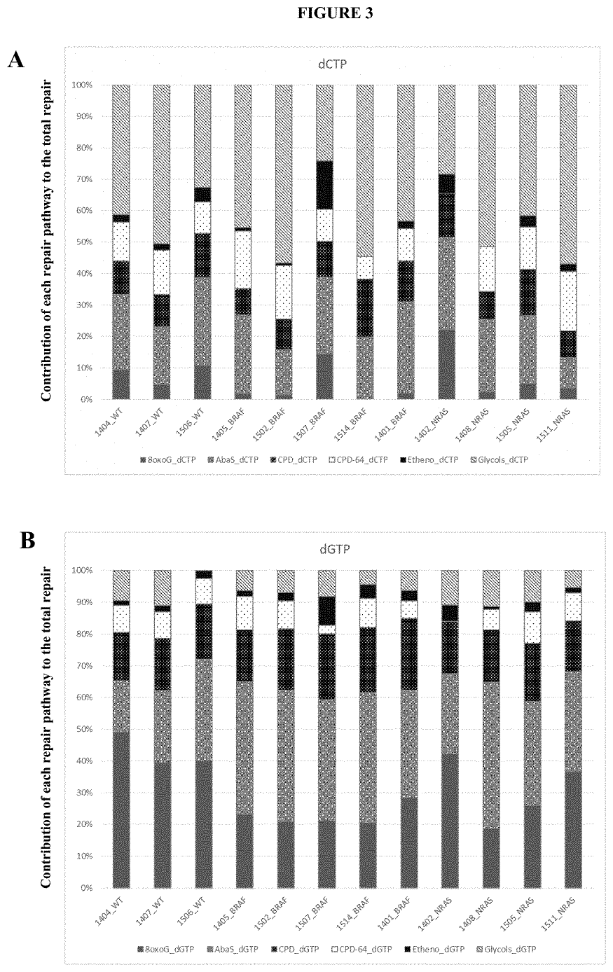

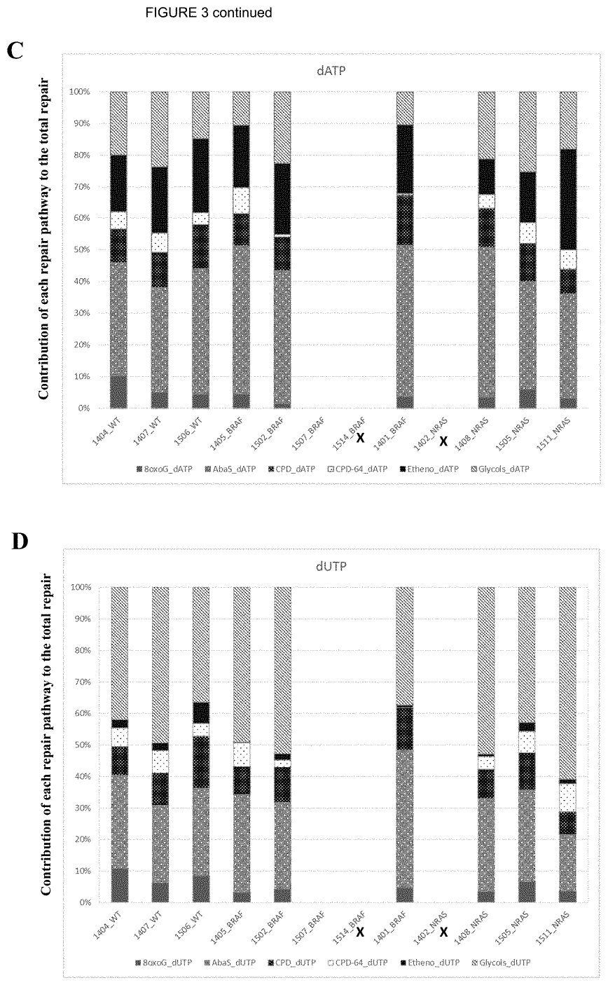

The contribution serves to determine the relative importance of the various repair pathways for each sample (FIG. 3A to 3D). The “contribution” signature allows a precise characterization of the effectiveness of each of the assayed repair pathways relative to the others. The contribution gives a parameter independent of the absolute value of the measured signals and is therefore complementary to the repair signature. The contribution varies for each sample depending on the nucleotide used. Each sample will be characterized by as many “contribution” profiles as nucleotides used. These “contribution” profiles may be compared to the “contribution” profiles from the reference library in order to verify the consistency of the classification.

For example, a sample may be in a certain “contribution” class from the library with the nucleotide X (corresponding to a subtype) and in another “contribution” class with the nucleotide Y, which discloses an inconsistency in the classification. This inconsistency indicates that the sample is different from other samples from the class and therefore might not respond to the therapy prescribed for this class.

The “contribution” profiles are closely associated with cellular signaling/proliferation pathways and functionality thereof.

The “contribution” profiles are closely associated with the capacities tumors to respond to chemotherapy and radiation therapy.

Based on the repair signature obtained from step c₁), the relative incorporation rate of each labeled nucleotide for at least two DNA lesions and independently for each DNA lesion (step c₃)) can also be determined.

For example in a repair reaction executed with four different labeled nucleotides (X, Y, Z and W), the relative incorporation rate of the nucleotide X for the repair of two lesions (first and second lesion) is determined independently for each of the lesions by applying the following formula:

Relative incorporation rate of the nucleotide X for the repair of the first lesion;

$$\% X = \frac{{I}_{X 1} - {I}_{C / X}}{Σ ( ( {I}_{X 1} - {I}_{C / X} ) + ( {I}_{Y 1} - {I}_{C / Y} ) + ( {I}_{Z 1} - {I}_{C / Z} ) + ( {I}_{W 1} - {I}_{C / W} ) )} \times 100$$

Relative incorporation rate of the nucleotide X for the repair of the second lesion;

$$\% X = \frac{{I}_{X 2} - {I}_{C / X}}{Σ ( ( {I}_{X 2} - {I}_{C / X} ) + ( {I}_{Y 2} - {I}_{C / Y} ) + ( {I}_{Z 2} - {I}_{C / Z} ) + ( {I}_{W 2} - {I}_{C / W} ) )} \times 100$$

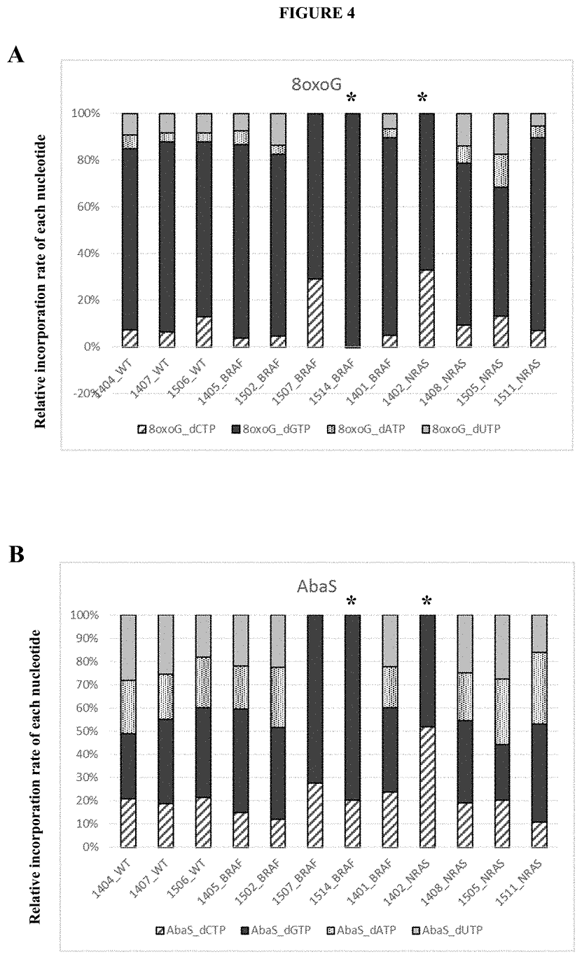

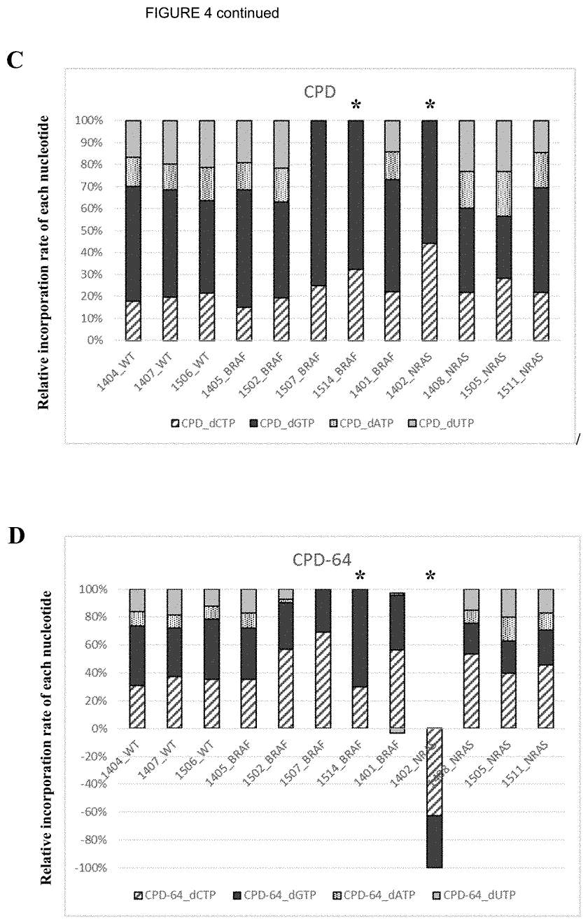

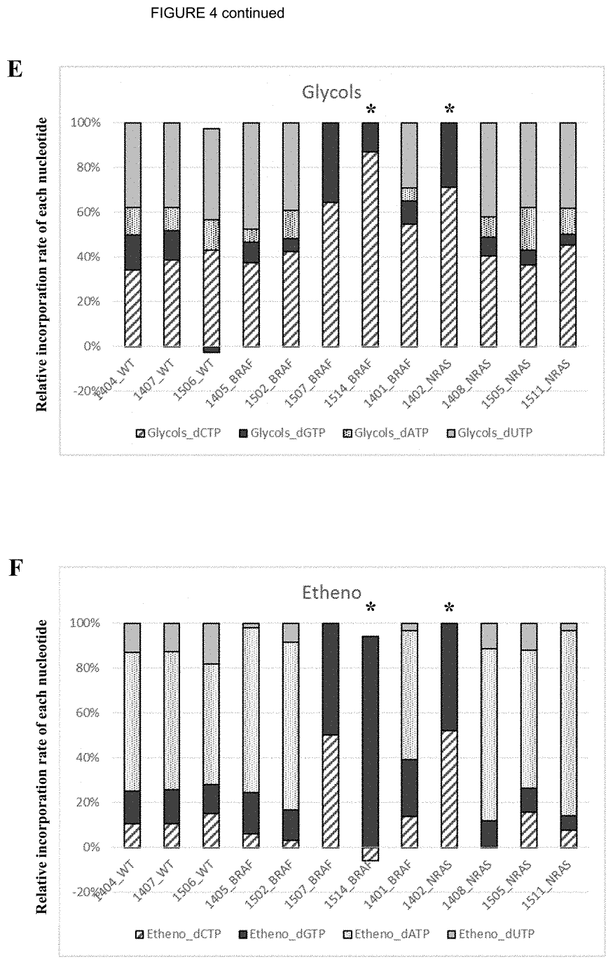

This calculation, shown in FIGS. 4A à 4D, is used to determine whether the repair mechanisms are functional in the samples, and in particular the step involving polymerases, really are those expected in connection with the reference library. The interest is in directly identifying the malfunctions responsible for mutations or for any other genomic defect supporting the appearance of neoantigens on the surface of tumors, associated with a better efficacy of immunological treatments. The direct detection of repair profiles and in particular the relative incorporation rate of each nucleotide avoids the use of PCR amplification methods necessary to the prediction of the neoantigens, which introduce biases (The problem with neoantigen prediction, Nat. Biotech., 2017, 35, 97). The tumors for which repair activities are absent or have malfunctions because of genomic mutations or defects could more favorably respond to immunotherapies.

Thus, the use of two nucleotides overcomes the disadvantages from the use of only one nucleotide which gives only a partial profile of the tumor and partial information on the functional repair pathways. In particular, the use of a single nucleotide does not make it possible to know what repair pathway is called on; what polymerase is involved; whether the repair is faithful; whether, if the signal is absent, another pathway could act as a replacement; it only allows a partial comparison with one reference library; it does not allow determination of what nucleoside is preferentially incorporated in the repair of each lesion.

Preferably, the method of the invention comprises the establishment of the isolated tumor cell repair profile from a tumor sample from a patient, and the comparison of said profile with the at least one reference profile, obtained for the same cancer subtype. The reference profile is established, simultaneously with or before the repair profile of the cells from the patient's tumor. Preferably, the comparison is done with a reference library comprising reference profiles of various subtypes of a cancer including at least the cancer subtypes of the patients to be tested, in particular the cancer subtypes which guide or condition the choice of treatments in the patients.

Preferably, the profile from step d) comprises:

the enzymatic signature of DNA repair of tumor cells;

the contribution of the repair of each DNA lesion independently for each labeled nucleotide compared to the total repair; and

the relative rate of incorporation of each labeled nucleotide for at least two DNA lesions, independently for each DNA lesion.

The repair signatures, the “contribution” profiles and the relative incorporation rates can be combined for comparison with the reference library processed under the same conditions.

The repair profiles can further be used for predicting the responses to chemotherapy and radiation therapy or for selecting patients responding to DNA repair inhibitors. The same repair profile can thus be used for choosing the best treatment from several classes of therapies or from combinations of therapies.

The method according to the invention is useful for choosing the best therapeutic option for the patient depending on the profile of DNA repair capacities of the cells from the patient's tumor, obtained by the method according to the invention. The best therapeutic option is chosen from several classes of therapies or from combinations of therapies.

The repair profiles obtained in step d) condition the treatment to be given to a patient:

If the repair profile of the tumor corresponds to the profile from the reference library for the same cancer types or subtypes, effective treatments for the samples classified in the same way in the reference library will be applied;

If the repair profile shows malfunctions in the DNA repair (e.g. some activities not present, polymerase defects, etc.), immunotherapies can be used;

The resulting repair profile can be used to choose a radiation therapy, or chemotherapy, or any other treatment damaging the DNA.

The repair profile can be used to choose a DNA repair inhibitor, for example for inhibiting a specific pathway at the origin of the resistance of the tumor to DNA targeting treatments, or else for inhibiting a redundant pathway, so as to create a synthetic lethality;

The repair profile can be used to choose different treatment combinations.

A high specific repair activity can be associated with a radiation resistance or chemoresistance. A specific repair inhibitor for this pathway can be used in this case. The inhibitor can be given, in combination therapy or not, with radiation therapy or chemotherapy creating lesions repaired by another pathway or the same pathway. Combinations of inhibitors of additional pathways can be considered for avoiding repair compensations due to the activation of redundant pathways.

On the other hand, low activity may be associated with radiation or chemotherapy sensitivity. Preferably a treatment will be selected which creates lesions repaired by the dysfunctional pathway.

When all repair activities are high, a targeted therapy can be used targeting signaling pathways regulating DNA repair, if an activating target is identified.

Tumors for which repair activities are missing or have malfunctions could be treated by immunotherapy.

In all cases, treatments from different classes (e.g. radiation therapy or chemotherapy, targeted therapy and immunotherapy) can be combined.

The object of the present invention is also a method for generating a profile of DNA repair capacities of tumor cells according to the invention for the cancer prognosis, choice, monitoring and/or the prediction of therapeutic efficacy of a cancer treatment in a patient, depending on the profile of DNA repair capacities of tumor cells obtained in step d).

The object of the present invention is a treatment method for a cancer in a patient comprising:

establishing the profile of DNA repair capacities of tumor cells coming from said patient according to the method of the invention;

selecting the most appropriate treatment for the patient depending on the resulting repair profile; and

administering the treatment to the patient.

The treatment is chosen in particular from chemotherapy, radiation therapy and immunotherapy. The chemotherapy directly or indirectly affects the DNA or the repair of the DNA.

The object of the present invention is a prognostic method for cancer in a patient comprising:

establishing the profile of DNA repair capacities of tumor cells coming from said patient according to the method of the invention;

determining the prognosis of the patient's cancer based on the resulting repair profile.

The patient's survival can in particular be determined with the prognostic method according to the present invention. In fact, there is a correlation between the intensity of the repair signal measured with the method for establishing the profile of DNA repair capacities according to the invention and patient survival. Preferably, the cancer is related to exposure to genotoxic agents, for example a melanoma, in particular metastatic melanoma of determined subtype, specifically, mutated BRAF, mutated NRAS or unmutated for BRAF and NRAS.

An object of the present invention is also the use of the use of the repair profile, for tumor cells isolated from a patient's tumor, obtained by a method for generating a profile of DNA repair capacities of tumor cells according to the invention, as biomarker of the cancer prognosis, choice, monitoring and/or prediction of the therapeutic efficacy of a cancer treatment in a patient.

The present invention also relates to reference libraries comprising profiles of characterized tumor repair capacities (reference profiles), corresponding to different subtypes of different cancers, obtained by the method for generating a profile of DNA repair capacities of tumor cells according to the invention.

Reference libraries containing reference profiles of various subtypes of various cancers including at least one of the cancer subtypes of the patients to be tested, in particular the cancer subtypes which guide or condition the choice of anticancer treatments in the patients. For each of these particular cancer subtypes, the reference bank includes the reference profile of patients responding or not responding to the recommended therapeutic treatment, in particular a targeted therapy, in particular therapy guided by mutations in key genes, like for example for melanoma the mutations in BRAF and NRAS genes.

An object of the present invention is also a kit for implementing the method according to the invention, comprising:

at least one damaged DNA comprising distinct lesions such as defined above, preferably damaged plasmids such as defined above, preferably immobilized on an appropriate support such as defined above; and at least two repair buffers;

a repair buffer comprising the agents necessary for the activity of the DNA repair enzymes present in the tumor cell extracts such as defined above and at least two different labeled nucleotides, in a single buffer or in separate buffers for each nucleotide

The present invention also relates to a method for classification of a cancer of defined type, from a patient's tumor sample, comprising at least the following steps:

i) establishing the profile of DNA repair capacities of tumor cells isolated from said tumor according to the method of the invention;

ii) comparing the profile of repair capacities obtained in the preceding step with a reference library containing profiles of repair capacities of various subtypes of the cancer; and

iii) determining the cancer subtype affecting the patient by similarity of the profile of repair capacities obtained from the patient with a reference profile of a cancer subtype from the reference library.

The present invention also relates to an antitumor agent screening method, comprising at least the following steps:

bringing tumor cells into contact with at least one agent to be tested;

establishing the profile of DNA repair capacities of the tumor cells treated by said agent and untreated tumor cells, by the method of the invention; and

selecting agents capable of modulating the DNA repair profile of said tumor cells. Preferably, the screening method is implemented with tumor cells in culture.

The antitumor agents selected by the screening method according to the invention are useful for treatment of cancers.

In addition to the preceding provisions, the invention further comprises other dispositions, which will emerge from the following description which refers to examples of implementation of the subject matter of the present invention which are in no way limiting, with reference to the attached drawings in which:

FIG. 1 shows the repair signatures of various DNA lesions by metastatic melanoma extracts, in presence of various labeled nucleotides. A. dCTP. B. dGTP. C. dATP. D. dUTP. 8oxoG: 8-oxo-guanine. AbaS: abasics sites. CPD: Cyclobutane type pyrimidine dimers CPD-64: a mixture of cyclobutane type pyrimidine dimers (CPD) and pyrimidines-pyrimidiones (6-4) (6-4 photoproducts or 6-4PP). Etheno: mixture of Etheno-guanines (Etheno-G) and Etheno-adenines (Etheno-A). Glycols: Mixture of thymine and cytosine glycols.

FIG. 2 shows the classification of repairs signatures by repair similarity. A. All data together. B. dCTP. C. dGTP. D. dATP. E. dUTP.

FIG. 3 shows the contribution from each repair pathway to the total repair for each of the labeled nucleotides. A. dCTP. B. dGTP. C. dATP. D. dUTP. X: not tested.

FIG. 4 shows the relative incorporation rate of each labeled nucleotide for the repair of each of the lesions. A. 8oxoG: 8-oxo-guanine. B. AbaS: abasic sites. C. CPD: Cyclobutane type pyrimidine dimers D. CPD-64: a mixture of cyclobutane type pyrimidine dimers (CPD) and pyrimidines-pyrimidiones (6-4) (6-4 photoproducts or 6-4PP). E. Glycols: Mixture of thymine and cytosine glycols. F. Etheno: mixture of Etheno-guanines (Etheno-G) and Etheno-adenines (Etheno-A). *: only dCTP and dGTP were used.

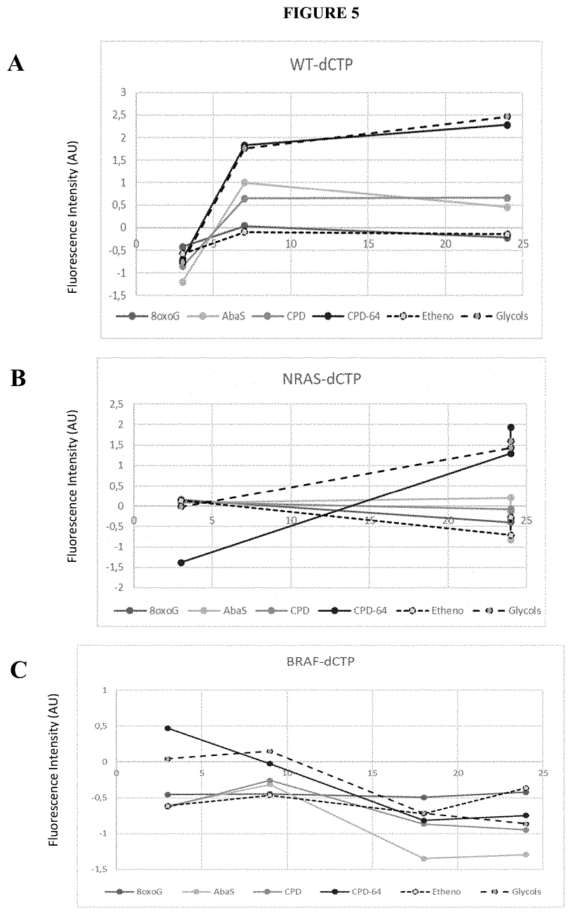

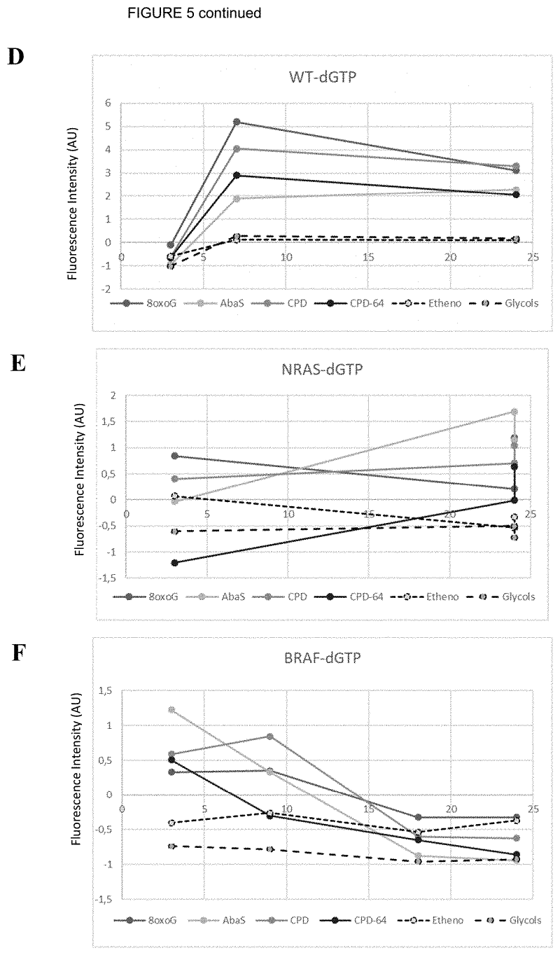

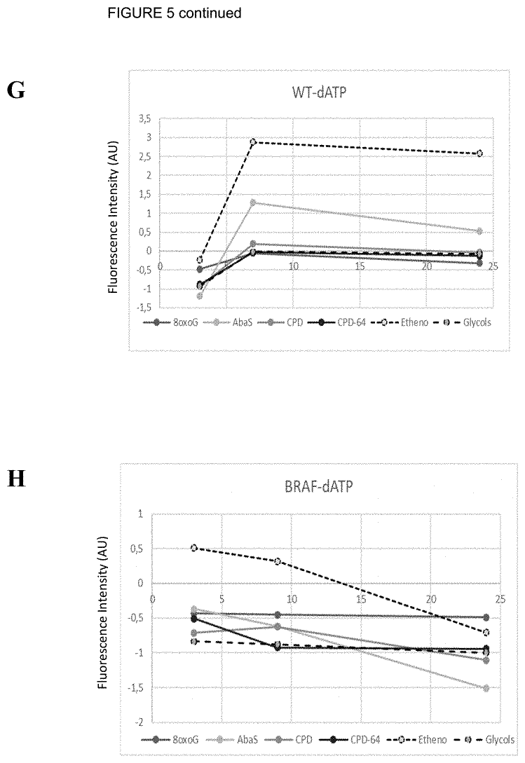

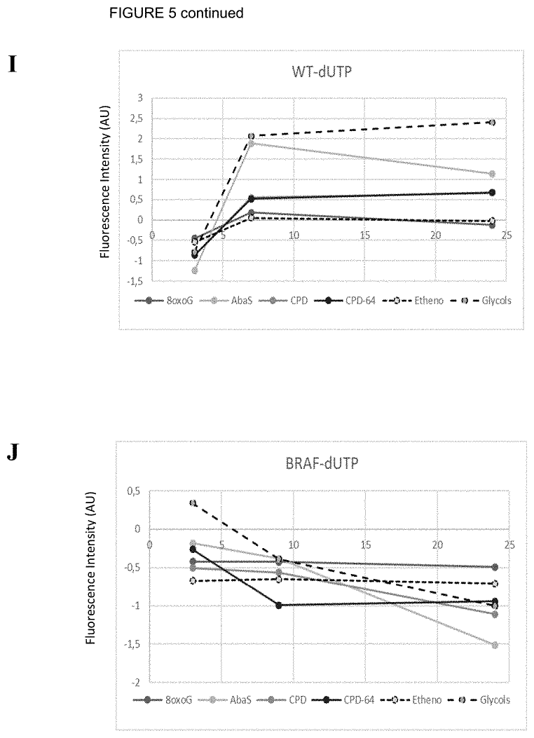

FIG. 5 shows the correlation between survival (abscissa) and intensity of the repair signal (ordinate) by mutation group and by dNTP. A. WT-dCTP. B. NRAS-dCTP. C. BRAF-dCTP. D. WT-dGTP. E. NRAS-dGTP. F. BRAF-dGTP. G. WT-dATP. H. BRAF-dATP. I. WT-dUTP. J. BRAF-dUTP.

### EXAMPLE: PRODUCTION OF REPAIR SIGNATURES AND REPAIR PROFILES FROM METASTATIC MELANOMA SAMPLES

### 1. Material and Methods

### 1.1 Patients

The samples (node or tumor) are from patients with a known metastatic melanoma subtype (BRAF; NRAS; WT (not mutated for BRAF and NRAS)) whose clinical data are presented in Table I.

The various treatment types administered to the study patients are shown in Table II below.

TABLE II
Treatment Administered to the Patients

| Name | Type |
|---|---|
| DERMA | Vaccine with melanoma specific antigen 3 |
| | MAGE-A3 antigen-specific cancer immunotherapeutic |
| | (ASCI) |
| Ipilimumab | anti CTLA-4 monoclonal antibody |
| Nivolumab | anti-PDL-1 monoclonal antibody |
| Pembrolizumab | anti PD-1 monoclonal antibody |
| Dabrafenib | mutated BRAF enzyme inhibitor |
| Vemurafenib | muted BRAF enzyme inhibitor |
| Trametinib | MEK enzyme inhibitor |
| RadioT | Radiation therapy |

### 1.2 Sample Preparation

The tumor or lymph node samples are collected by surgical resection and then prepared as follows.

### a. Cellular Dissociation

The cellular dissociation is performed starting from a biopsy of metastatic melanoma lymph nodes or tumors. The biopsy, about 5 mm³, is placed in a 100 mm diameter petri dish with a few drops of RPMI-1640 Glutamax (Life Technologies). It is then cut in fragments using a scalpel. These fragments are transferred to a 15 mL centrifuge tube containing 5 mL of digestion medium (1 mg/mL Collagenase D (Roche), 50 IU/mL DNase I (Roche), RPMI-1640 Glutamax (Life Technologies)). The tube containing the fragments is incubated 30 minutes at 37° C. in an oven and the fragments are returned to suspension every 10 minutes using a 10 mL pipette. At the end of the incubation, a volume of 2.5 mL of digestion medium stored at room temperature is added. The fragments are again incubated for 30 minutes at 37° C. in an oven and returned to suspension every 10 minutes using a 10 mL pipette. At the end of incubation, the tube is centrifuged at 1200 RPM for 10 minutes at 4° C. The supernatant is eliminated and the pellet is taken up in 10 mL of cold PBS-EDTA buffer (10 mM EDTA (Sigma), 1X D-PBS (Life Technologies)). The cells are then deposited on a 70 um cellular sieve (Dutcher), positioned on a 50 mL centrifuged tube stored in ice. The 15 mL tube is rinsed by adding an additional 5 mL of cold PBS-EDTA buffer and the residual fragments are passed through the sieve. A sample is taken in order to count the cells. The 50 mL tube is centrifuged at 1200 RPM for 10 minutes at 4° C. The supernatant is eliminated and the pellet is taken up in an Albumin/DMSO (90% Human Albumin, 10% DMSO (Sigma)) cryopreservation solution at a rate of 4×10⁶ cells per cryo-tube. The cells are progressively frozen at −80° C. in a freezer container and then transferred to liquid nitrogen for long-term storage.

### b. Preparation of Nuclear Extracts

The tumor cells are thawed at ambient temperature, and then centrifuged at 500 RCF (Relative Centrifugal Force) for five minutes of 4° C. The supernatant is eliminated and the cells washed with 1 mL of cold PBS and then re-centrifuged at 500 RCF for five minutes at 4° C. The cells are taken up in 1 mL of buffer A (10 mM HEPES pH 7.8, 1.5 mM MgCl₂, 10 mM KCl, 0.02% Triton X-100, 0.5 mM DTT, 0.5 mM PMSF (phenylmethylsulfonyl fluoride) and incubated for 10 minutes in ice. Each tube is vortexed for 30 seconds. The cells are centrifuged five minutes at 2300 RCF, the supernatant is eliminated, and then the cells are taken up and 30 μL of buffer B (10 mM HEPES pH 7.8, 1.5 mM MgCl₂, 400 mM KCl, 0.2 mM EDTA, 25% glycerol, 0.5 mM DTT, 0.5 mM PMSF, 100 μL antiproteases 0.7 X (Complete-mini, Roche). After 20 minutes incubation over ice, lysis of the nuclear membrane is done by two freeze/thaw cycles in liquid nitrogen at −180° C. and 4° C. The tubes are centrifuged at 16,000 RCF for 10 minutes at 4° C. The supernatant is distributed in 10 μL aliquot portions. A fraction of the supernatant is collected for proteomic assay by using the micro-BCA method (Interchim). The aliquot portions undergo rapid freezing over 30 seconds in liquid nitrogen at −180° C. and then are stored at −80° C.

### 1.3 Analysis of DNA Enzymatic Repair Activities

The analysis was done on chips comprising lesioned plasmids, prepared as previously described (Millau et al., Prunier et al., op. cit.).

The reaction mixture is prepared for each dNTP-biotin, so as to get DNA repair enzymatic signatures in the presence of various dNTP labeled with biotin. The reaction lasted three hours at 30° C. in reaction chambers filled with 12 μL of medium made up of 4.8 μL of 5X buffer (200 mM Hepes KOH pH 7.8, 35 mM MgCl₂, 2.5 mM DTT, 1.25 μM of each unlabeled dNTP (Perkin Elmer), 17% Glycerol, 50 mM phosphocreatine (Sigma), 10 mM EDTA, 250 μg/mL creatine phosphokinase, 0.5 mg/mL BSA, 0.5 μL ATP 100 mM (Amersham) and 1.25 μM of dNTP-biotin (Perkin Elmer)), in the presence of nuclear extracts at a final concentration of 0.2 mg/mL, with the solution topped off to 24 μL with H₂). Each sample is tested on two chips (in two reaction chambers) for each of the dNTP-biotin.

After incubation, the slides are rinsed twice for three minutes with PBS/Tween 0.05%, and then twice for three minutes with MilliQ water. The slides are incubated in a 225 ng/mL bath of Streptavidine-Cy5, in the presence of 0.1 mg/mL of BSA for 30 minutes at 30° C. The slides are rinsed twice for three minutes with PBS/Tween 0.05%, and then twice for three minutes with MilliQ water. Finally, the slides are centrifuged for 30 seconds and then dried for 10 minutes at 30° C. The slides are scanned at 635 nm (Innoscan Innopsys) for quantification of the fluorescence. The fluorescence value of the control plasmid (no lesions) is subtracted from the total intensity value obtained for each lesion. The data are normalized as described in Millau et al., op. cit. The data are expressed in Fluorescence Intensity (Arbitrary Units) and standard deviation for each sample and each of the dNTP tested.

### 1.4 Statistical Analyses

The Fluorescence Intensity values obtained are centered-reduced, meaning that their average is brought to 0 and the standard deviation to 1.

The data are then analyzed by hierarchical classification such as described in the Forestier et al publication, by using the Euclidean distance (PLoS One. 2012; 7:e51754).

### 2. Results

### 2.1 Repair Signatures

The method according to the invention was implemented on metastatic melanoma samples (lymph nodes or tumors) for patients with known BRAF and NRAS gene mutation status (Table I). The repair reactions were done with 0.2 mg/mL of nuclear protein extract in duplicate for three hours at 30° C. The fluorescence intensities were measured with a scanner in order to quantify the level of incorporation of each triphosphate nucleoside in each plasmid. The fluorescence intensities obtained with the control plasmid are subtracted (I_{X1}−I_{c}).

### Repair Signatures with the Various DNTP

The different labeled nucleotides reveal different distribution value profiles for a single lesion for each mutation group (FIGS. 1A to 1D) allowing to thus get more precise information on the specific distribution mechanisms.

BRAF and NRAS mutations affect the DNA repair signature at different levels (overall and specific activities). In particular, the samples with the mutated BRAF gene show a very low repair activity compared to wild-type.

### Classification of Repair Signatures, All Data Combined

The data are then centered-reduced (average to 0; standard deviation to 1) for each lesion, so as to allow unbiased classification.

Next, a hierarchical classification is done with Euclidean distance. The samples, in sufficient number, serve both to make up the reference library comprising the classification of the repair signatures depending on the mutations (e.g. BRAF, NRAS), and to determine the differences of each of the samples compared to the theoretical class thereof.

The samples are classified by repair signature similarity. The results show that the various dNTPs determine different classes up to a few exceptions (FIG. 2A). This indicates that each dNTP provides distinct and complementary information about the samples.

In particular, when all the results are considered and classified together, it is seen that the samples analyzed with dCTP remain grouped but separated by mutation group. This shows that the BRAF and NRAS mutations exert a dominant effect on the DNA repair signature.

This confirms that the analysis by dCTP shows that mutations in the signaling pathways (MAP Kinase) impact the DNA repair signature. This information is important and places a particular role for the analysis by dCTP, especially if the treatment applied is the targeted therapy (guided by the BRAF and NRAS mutations).

When dGTP is used, the WT are on one side, and the mBRAF and mNRAS are in another class. Two categories of mBRAF samples are identified: a first group has a weak repair activity and the second group, combined with the mNRAS, has a high repair activity (FIG. 2C).

The results obtained with dATP and especially dUTP are more fragmented.

The individual analyses by dNTP provide additional information (FIGS. 2B, C, D, E).

### Classification of Repair Signatures Obtained with Labeled dCTP

The use of dCTP discriminates the mutated BRAF from other sample categories (FIG. 2B). All the BRAF samples are in a shared class. It is however separated into two subclasses, which distinguishes two categories of mutated BRAF samples (Class 1.1 containing 1507 and 1514; and Class 1.2 containing 1405, 1502 and 1401).

Class 1.1 corresponds to patients who have the longest survival.

The samples 1404_WT and 1407_WT are classified together (FIG. 2B).

In contrast, the sample 1506_WT is classified with the majority of mutated BRAF samples (FIG. 2B). It therefore has a profile similar to the mutated BRAF samples from Class 1.1 (1507 and 1514) and not to the WT samples.

The mutated NRAS samples are in a shared group (FIG. 2B). However the sample 1402_NRAS is different; it is part of a specific profile (FIG. 2B) which has a very low survival.

### Classification of Repair Signatures Obtained with Labeled dGTP

For dGTP, as for dCTP, the samples 1404_WT and 1407_WT are classified together which confirms the similarity of their profile (FIG. 2C). Similarly, 1506_WT is classified separately, with the mutated BRAF samples (1502, 1507 and 1514), (FIG. 2C).

All the mutated BRAF and mutated NRAS samples are classified in a single group, separated into two subgroups (FIG. 2C). The samples 1405_BRAF and 1401_BRAF are close to mutated NRAS in this classification (FIG. 2C).

dGTP distinguishes between the WT samples for BRAF and NRAS and the mutated samples for BRAF and NRAS; by using this nucleotide, the samples having activated pathways can be distinguished from samples having inactivated signaling pathways.

### Classification of Repair Signatures Obtained with Labeled dATP

For dATP, as for dCTP and dGTP, the samples 1404_WT and 1407_WT are classified together which confirms the similarity of their profile (FIG. 2D).

All the mutated BRAF are classified in the same group, which again includes 1506_WT, confirming that the sample has a profile similar to the mutated BRAF samples (FIG. 2D).

The use of dATP does not discriminate between the two WT (1404 and 1506) of the NRAS (1505 and 1511) except for 1408_NRAS which shows a specific profile (FIG. 2D).

### Classification of Repair Signatures Obtained with Labeled dUTP

For dUTP, as for dCTP, dGTP and dATP, the samples 1404_WT and 1407_WT are classified together which confirms the similarity of their profile (FIG. 2E).

The use of dUTP reveals a similarity of the samples 1404_WT and 1407_WT with 1505_NRAS (FIG. 2E).

The set of the other samples is in a class divided in two, where the mutation groups are mixed but where 1506_WT is found with the mutated BRAF and in particular 1507_BRAF, 1502_BRAF and 1401_BRAF (FIG. 2E).

As with dGTP, the 1405_BRAF sample is close to mutated NRAS samples in this classification made from the dUTP data (FIG. 2E).

In conclusion, despite the recognized genomic diversity of metastatic melanomas, the method according to the invention brings out repair type signatures for each of the mutations in the BRAF or NRAS genes and in the group not comprising mutations in these genes.

By using various labeled dNTPs, profile similarities can be identified which cannot be determined on the basis of mutations alone and classes can be established beyond the classification by mutation type, which is not sufficient for predicting the response to therapies. A combined analysis of the results obtained with the various dNTPs reveals unexpected information and brings out new subgroups.

The dNTP by dNTP classifications identify mismatching patients for the classification of subtypes by the detection of mutations and consequently identifies dysfunctions in the regulation of DNA repair by the signaling pathways. This information can be used in the case of prescriptions for targeted therapy, for which it is the mutations in the key genes like BRAF and NRAS which guide the prescription.

The various repair profiles observed in each subtype of melanoma (WT, mutated BRAF, NRAS) are compared with the repair profiles from the reference library in order to determine the profiles which correspond to patients responding to the targeted therapies. This comparison is thus used to administer the right treatment to the right group of patients.

### 2.2 Contribution of Each Repair Pathway to the Total Repair

For the calculation of the contribution, the negative data are adjusted to 0, specifically ((I_{X1}-I_{c})=0, if negative). This parameter qualifies the specific repair activities relative to each other for a single sample, and identifies the missing activities.

By comparison with the reference library made up of cancer types and/or subtypes, it identifies specific repair activities out of line (increased or reduced or absent) compared to the others.

### dCTP Label

The analysis of the contribution of each repair pathway to the total repair, for the repair reactions done with dCTP, brings out a difference between the mutated BRAF samples and the other categories, mainly for the repair of 8oxoG and the Ethenobases (Etheno), (FIG. 3A). The BRAF samples have very specific profiles compared to the other NRAS or WT samples.

As it relates to the 8oxoG, for 4 out of 5 samples, the relative incorporation of dCTP is either weaker than for the other samples (for 1405, 1502 and 1401) or zero (for 1514).

It is the same for the incorporation of dCTP for the repair of Etheno (reduced for 1405 and 1502, zero for 1514).

Inversely for the sample 1507, the incorporation of dCTP is increased compared to the other samples for the repair of 8oxoG and Etheno and relatively weak for Glycols; which distinguishes this mutated BRAF sample from the others.

### dGTP Label

For the “contribution” analysis, the dGTP label serves to distinguish the WT samples from the other samples essentially in the level of incorporation thereof for the repair of 8oxoG, which is in general higher (FIG. 3B).

Inversely, the mutated BRAF are distinguished from other samples by a lower dGTP incorporation level with 8oxoG (FIG. 3B).

With this label, note that the Glycols are not repared for 1506_WT, which distinguishes this sample from two other WT (FIG. 3B).

It is also noted that with this label there are no repairs of the CPD-64 for 1402_NRAS (FIG. 3B), which has a very low survival.

### dATP Label

For the two BRAF samples where it is measurable (1502 and 1401), the contribution of dATP to the repair of CPD-64 is very small compared to other samples (FIG. 3C)._This label is not incorporated by the 1507_BRAF sample, whatever the lesion considered (FIG. 3C).

### dUTP Label

This label is not incorporated by the sample 1507_BRAF, whatever the lesion considered (FIG. 3D). The contribution of dUTP to the repair of CPD-64 is zero for the BRAF 1401 sample (FIG. 3D).

In conclusion, the calculations of the contribution bring out specific profiles which characterize the samples independently from the fluorescence intensity. In this way the relative importance of each repair pathway can be qualitatively compared against all the functional repair activities assayed at the same time.

Despite the recognized genomic diversity of metastatic melanomas, repair type profiles can be associated with each of the mutations of the BRAF or NRAS genes and in the group not comprising mutations in these genes. Thus it is necessary to take this information into consideration when seeking to identify all of the repair defects.

### 2.3 Shows the Relative Incorporation Rate of Each Nucleotide for the Repair of Each of the Lesions

For this calculation it is necessary to form a combination of values that were obtained independently by performing repair reactions in the presence of different dNTPs.

For each sample assayed, the contribution of each labeled dNTP to the resulting total fluorescence is examined for the set of four dNTPs or two dNTPs (in some examples). The resulting information supplements the repair signature profile and the “contribution” profile. The samples 1514_BRAF and 1402_NRAS were characterized solely with labeled dCTP and labeled dGTP. All the other samples were characterized with the four labeled dNTP. The results are shown in FIGS. 4A to 4F.

dGTP is the majority nucleotide incorporated for the repair of 8oxoG (FIG. 4A). For the sample 1505_NRAS, it is noted that other nucleotides are relatively significantly incorporated, which indicates the involvement of alternative repair pathways, or errors of the polymerases acting in the patch repair and distinguishes this sample (FIG. 4A).

The sample 1507_BRAF is distinguished from other samples for the repair of abasic sites (FIG. 4B).

If no defect in the repair is present, for the repair of a given lesion, an identical contribution of each dNTP is expected. In fact, for a given lesion, the repair systems, because of their specificity, should be identical between samples. However, disparities between the samples are observed, which discloses defects of the repair. For each mutation group, the profiles not conforming to the expected profile can signal alterations of one or more repair mechanisms which leads to the incorporation of some nucleotides in the place of the expected ones, and supports the appearance of mutations.

### 2.4 Correlation between the Repair Signature and Patient Survival

The capacity of the enzymatic repair signature to predict the survival was analyzed from reduced, centered data combining the patient deceased 18 months after collection of the tumor sample (1514) and patients not deceased after 24 months (M24); patients lost from sight were not incorporated in the study. Four survival groups were defined:

M3: deceased 3 months post-collection

M7: deceased 7 months post-collection

M9: deceased 9 months post-collection

M18: deceased 18 months post-collection

M24: survived at least 24 months post-collection.

Results are to be distinguished according to the mutation group considered:

WT Patients for BRAF and NRAS- dCTP: A very poor survival is associated with very low DNA repair capacities (FIG. 5A). The most discriminating repair measurements are CPD-64, Glycols, AbaS and CPD (FIG. 5A).

dGTP: Just as for dCTP, a very poor survival is associated with very low DNA repair capacities (FIG. 5D). The most discriminating repair measurements are 8oxoG, CPD CPD-64 and AbaS (FIG. 5D).

dATP: As with the preceding nucleotides, a very poor survival is associated with very low DNA repair capacities (FIG. 5G). Two lesions are discriminating: Etheno and AbaS (FIG. 5G).

dUTP: As with the preceding nucleotides, a very poor survival is associated with very low DNA repair capacities (FIG. 5I). Two lesions are discriminating: Glycols and AbaS (FIG. 5I).

### Mutated BRAF Patients

dCTP: Survival is inversely proportional to some DNA repair activities (FIG. 5C). In particular, an inverse linear relation is observed between the CPD-64 repair level and survival. For patients deceased 3 or 9 months post-collection, the repair level is greater than for patients deceased or still living at 18 or 24 months for CPD-64, AbaS, CPD, and Glycols (FIG. 5C).

dGTP: An inverse linear relationship is observed between the repair of some DNA repair activities, in particular AbaS, and survival (FIG. 5F). For patients deceased 3 or 9 months post-collection, the repair level is greater than for patients deceased or still living at 18 or 24 months for AbaS, 8oxoG, CPD, and CPD-64 (FIG. 5F).

dATP: An inverse linear relation is observed between the AbaS, Etheno repair level and survival (FIG. 5H). For the patient's deceased 3 months post-collection, the repair level is greater than for patients deceased or still living at 24 months for AbaS, Etheno, CPD and CPD-64 (FIG. 5H).

dUTP: An inverse linear relation is observed between the Glycol, AbaS repair level and survival (FIG. 5J). For patients deceased 3 months post-collection, the repair level is greater than for patients deceased or still living at 24 months for Glycols, AbaS, CPD, and 8oxoG.

### Mutated NRAS Patients

dCTP: A proportional relationship is observed between survival and DNA repair level for CPD-64 and Glycols, and an inverse relationship is observed for Etheno and 8oxoG (FIG. 5B).

dGTP: A proportional relationship is observed between survival and DNA repair level for CPD-64 and AbaS, and an inverse relationship is observed for Etheno and 8oxoG (FIG. 5E).

TABLE III
Correlation between Intensity of the
Repair Signal and Patient Survival

| | WT | | BRAF | | NRAS | |
|---|---|---|---|---|---|---|
| dCTP | Glycols | Correl | Glycols | Anti | Glycols | Correl |
| | CPD-64 | | CPD-64 | Correl | CPD-64 | |
| dGTP | CPD | Correl | CPD-64 | Anti | CPD-64 | Correl |
| | 8oxoG | | AbaS | Correl | AbaS | |
| | CPD-64 | | | | | |
| dATP | Etheno | Correl | Etheno | Anti | NA | |
| | AbaS | | AbaS | Correl | | |
| dUTP | Glycol | Correl | Glycol | Anti | NA | |
| | AbaS | | AbaS | Correl | | |
| | | | CPD | | | |

*Correl: Positive correlation
Anti-Correl: Negative correlation
NA: not-applicable

This study shows that the repair signature predicts patient survival; some activities are more predictive than others (FIG. 5). Interestingly, the relation between repair signal intensity and patient survival varies depending on the group of mutations (Tableau III).

TABLE I
Clinical Data about the Patients Tested

| | | | | Sample | | Initial | Treatment |
|---|---|---|---|---|---|---|---|
| Sample | Gender | Page | PhotoType | type | Mutation | treatment | at M3 |
| 1401 | M | 42 | III. | Scapular | BRAF | Derma | Vemurafenib |
| | | | | node-Skin-trunk | | | |
| 1402 | M | 32 | II | Node-trunk | NRAS | Ipilimumab | Pembrolizumab |
| | | | | | | Pembrolizumab | |
| 1404 | F | 62 | III. | Node-sub | WT | | Ipilimumab |
| | | | | clavicular | | | |
| 1405 | M | 50 | II | Arm | BRAF | Vemurafenib | M3 |
| | | | | metastases | | Dabrafenib | |
| 1407 | M | 69 | III. | Cerebral | WT | Radiation | Radiation |
| | | | | metastases | | therapy | therapy + |
| | | | | | | | Ipilimumab |
| 1408 | M | 60 | III. | Sub-clavicular | NRAS | | |
| | | | | node | | | |
| 1502 | F | 50 | III. | Cervical | BRAF | | |
| | | | | node | | | |
| 1505 | F | 83 | II | Inguinal | NRAS | | |
| | | | | node | | | |
| 1506 | M | 78 | II | Node | WT | | M3 |
| 1507 | M | 46 | III. | Abdominal | BRAF | | |
| | | | | node | | | |
| 1511 | M | 64 | III. | Inguinal | NRAS | | |
| | | | | node | | | |
| 1514 | M | 50 | III. | Node | BRAF | | |

| | Treatment | Treatment | Treatment | Treatment | |
|---|---|---|---|---|---|
| Sample | at M6 | at M9 | at M18 | at M24 | Death |
| 1401 | Nivolumab | M9 | | | M9 |
| 1402 | | | | | M3 |
| 1404 | Nivolumab | M7 | | | M7 |
| 1405 | | | | | M3 |
| 1407 | Nivolumab | Nivolumab | Nivolumab | Nivolumab | Not |
| | | | | | deceased |
| | | | | | at M24 |
| 1408 | Nivolumab | Nivolumab | Pembrolizumab | Ipilimumab | Not |
| | | | | | deceased |
| | | | | | at M24 |
| 1502 | | | | | Lost from |
| | | | | | view M3 |
| 1505 | | | | | Lost from |
| | | | | | view M3 |
| 1506 | | | | | M3 |
| 1507 | | | | | Thyroid |
| | | | | | cancer |
| | | | | | Not |
| | | | | | deceased |
| | | | | | at M24 |
| 1511 | Pembrolizumab | Pembrolizumab | Pembrolizumab | Nivolumab | Not |
| | | | | | deceased |
| | | | | | at M24 |
| 1514 | | Dabrafenib + | | RadioT | M18 |
| | | trametinib | | | |

1-11. (canceled)

12. An in vitro method for generating a profile of DNA repair capacities of tumor cells, comprising at least the following steps:
a) incubating a tumor cell extract comprising a DNA repair activity with at least two damaged DNA molecules comprising distinct DNA lesions and at least two different labeled nucleotides;
b) measuring the quantity of each labeled nucleotide incorporated in each damaged DNA molecule resulting from the activity of the repair enzymes present in said cellular extract in step a);
c) determining, from the values measured in step b), at least one of the following parameters:
c1) the enzymatic signature of the DNA repair;
c2) the contribution of the repair of each DNA lesion independently for each labeled nucleotide compared to the total repair; and
c3) the relative rate of incorporation of each labeled nucleotide for at least two DNA lesions and independently for each DNA lesion; and
d) establishing the profile of DNA repair capacities of said tumor cells based on the parameters determined in step c).

13. The method according to claim 12, wherein said tumor cells are isolated from samples of different previously characterized tumors, and at least one reference library comprising reference profiles of various subtypes of a cancer including at least the cancer subtypes of the patients to be tested are obtained.

14. The method according to claim 12, wherein the tumor is a metastatic melanoma for which the mutational status of the BRAF and NRAS genes is determined.

15. The method according to claim 12, wherein step a) is performed with:
at least one first DNA molecule comprising a single type of lesion repaired by the base excision repair system selected from the group consisting of: 8-oxo-G; abasic sites; Ethenobases; thymine and/or cytosine glycols, and
at least one second DNA molecule comprising a type of lesion repaired by the nucleotide excision repair system chosen from photoproducts.

16. The method according to claim 12, wherein said damaged DNA molecules are supercoiled plasmids.

17. The method according to claim 12, wherein the different labeled nucleotides comprise at least labeled dGTP and dCTP nucleotides.

18. The method according to according to claim 12, wherein the profile of step d) comprises:
the enzymatic signature of the DNA repair,
the contribution of the repair of each lesion independently for each labeled nucleotide compared to the total repair; and
the relative rate of incorporation of each labeled nucleotide for at least two DNA lesions, independently for each DNA lesion.

19. The method according to claim 12, wherein the profile of step d) is compared with a reference library.

20. A method for cancer prognosis in a patient, comprising generating a profile of DNA repair capacities of the patient tumor cells according to the method of claim 12 and establishing the prognosis of the patient based on the profile of DNA repair capacities of the patient.

21. A method for the choice, monitoring and/or prediction of therapeutic efficacy of a cancer treatment in a patient, comprising generating a profile of DNA repair capacities of the patient tumor cells according to the method of claim 12 and administering a cancer treatment to the patient based on the profile of DNA repair capacities of the patient.

22. A reference library containing metastatic melanoma samples of mutated BRAF, mutated NRAS, or non-mutated for BRAF and NRAS and the profiles of repair capacities of the various subtypes of said cancer obtained by the method according to claim 12.

23. A method for classification of a cancer of a defined type, comprising at least the following steps, starting from a patient tumor sample:
i) establishing the profile of DNA repair capacities of tumor cells isolated from said sample according to the method of claim 12;
ii) comparing the profile of repair capacities obtained in the preceding step with a reference library containing profiles of repair capacities of various subtypes of said cancer type; and
iii) determining the cancer subtype affecting the patient by similarity of the profile of repair capacities obtained from the patient with a reference profile of a cancer subtype from the reference library.

24. The method according to claim 13, wherein said cancer subtypes guide the treatment choice for the patients.

25. The method according to claim 15, wherein the Ethenobases are Etheno-guanines and/or Etheno-Adenines.

26. The method according to claim 15, wherein the photoproducts comprise a mixture of cyclobutane type pyrimidine dimers and pyrimidines-pyrimidiones (6-4).

27. The method according to claim 16, wherein the supercoiled plasmids are immobilized on a solid support.

28. The method according to claim 17, wherein the different labeled nucleotides comprise labeled dCTP, dGTP, dATP and dUTP nucleotides.
